(19) United States
(12) Patent Application Publication
(10) Pub. No.: US 2025/0099118 A1
(43) Pub. Date: Mar. 27, 2025

(54) DEVICE AND SYSTEM FOR THE TREATMENT OF ALZHEIMER'S DISEASE

(71) Applicant: MTS Medical AG, Konstanz (DE)

(72) Inventors: Axel Erich Theuer, Kirchberg (DE); Manfred W. Gekeler, Konstanz (DE); Nikolaus Hopfenzitz, Konstanz (DE); Hans-Joachim Raida, Konigswinter (DE)

(73) Assignee: MTS Medical AG, Konstanz (DE)

(21) Appl. No.: 18/892,777

(22) Filed: Sep. 23, 2024

(30) Foreign Application Priority Data
Sep. 22, 2023 (EP) 4342394

(51) Int. Cl. A61B 17/22; A61B 17/00; G16H 10/60; G16H 20/40

(52) U.S. Cl. CPC A61B 17/22022; G16H 10/60; G16H 20/40; A61B 2017/00194; A61B 2017/00225; A61B 2017/00477; A61B 2017/00716; A61B 2017/22025

(57) ABSTRACT

Devices (10) for extracorporeal shock wave therapy, a system for the treatment of Alzheimer's disease, a method for providing setting parameters for a treatment of Alzheimer's disease, a computer program and a computer program product are provided. The device (10; 20; 80; 90) for extracorporeal shock wave therapy, in particular for the treatment of Alzheimer's disease, namely an electrohydraulic shock wave device includes at least one shock wave generator (12) and at least one shock wave applicator (3). The device (10) has a capacitor discharge resonant circuit whose discharge frequency distribution has a maximum which is below 350 kHz, preferably below 300 kHz, more preferably below 250 kHz, still more preferably below 200 kHz, still more preferably below 150 kHz, still more preferably below 100 KHz.

### CROSS-REFERENCE TO RELATED APPLICATIONS

This application claims priority to European Patent Application No. 4342394, filed Sep. 22, 2023, which is incorporate herein by reference as if fully set forth.

### TECHNICAL FIELD

The invention relates to devices for extracorporeal shock wave therapy, a system for the treatment of Alzheimer's disease, a method for providing setting parameters for a treatment of Alzheimer's disease, a computer program and a computer program product.

In particular, the invention discloses a device and arrangement for the extracorporeal treatment of Alzheimer's disease (AD, Alzheimer's disease) in alternating fields and describes the control of the device.

Alternating mechanical fields, especially shock waves, are used which propagate through the skull with low loss via low-frequency bands.

The invention also includes a component for combined treatment in alternating mechanical fields (AMF) and alternating electric fields (AEF).

### BACKGROUND

The state of the art in extracorporeal shock wave therapy includes powerful electrohydraulic shock wave devices that were originally developed for kidney stone fragmentation (lithotripsy). These electrohydraulic shock wave generators have a (shock wave) applicator that can be attached to a shock wave device with a plug.

Electrohydraulic shock wave devices comprise an electrohydraulic shock wave generator with a discharge capacitor and an electrohydraulic shock wave applicator with a therapy head.

Between the shock wave generator and the actual therapy head, a cable can be arranged in the form of a tubular connection, which can contain current-carrying lines, e.g. coaxial high-voltage cables as well as control lines and possibly also fluid lines.

The cable can be permanently connected to the therapy head, and thus belong to the applicator, and connected to the shock wave generator via a detachable plug connection.

In electrohydraulic shock wave devices, a liquid-filled reflector and a pair of electrodes (inner conductor and outer conductor, each with a different electrical potential) are arranged in the therapy head. A hot plasma channel is generated in the liquid, e.g. water, by an applied high voltage during a discharge at the electrodes via a developing ion current, which leads to an exploding gas bubble, resulting a priori in a shock wave with a very high amplitude or intensity and an extremely short rise time.

The positive maximum pressure amplitude (p+) is significantly higher than the negative minimum pressure amplitude (p−) in the transient pressure curve. Reflectors can be used to focus the electrohydraulically generated shock wave onto the treatment zone. Due to non-linear effects during propagation, the shock wave can also split further or maintain the high pressure gradient during propagation. The shock wave propagates at a wave velocity that can be higher than the local sound velocity of the liquid in the reflector at the start of propagation. In this case, the explosion is also referred to as a detonation.

The following two types of electrohydraulic shock wave devices can be distinguished. In the first type, a charged capacitor is discharged via the electrodes using a switch. In the second type, the electrode gap is set so that discharge is possible without switching from a certain capacitor voltage. This type of shock wave device is referred to as an “electrohydraulic shock wave device with self-ignition”. The technologies described in this patent specification can be used in particular in the field of electrohydraulic shock wave devices with auto-ignition.

When pressure waves are generated electromechanically, i.e. electromagnetically or piezoelectrically, the wave trains always propagate at the local wave speed of the medium. This is due to the fact that electromagnetic or piezoelectric devices generate mechanical pressure waves in a fluid via vibrating surfaces or membranes, which split and overlap to form a shock wave in a narrow treatment area (focus area) on their way to the focus point or in the focus point. Due to the inertia of these electromechanical systems, they cannot—a priori—be used to generate shock waves, but only pressure waves that propagate at the local speed of sound of the medium. Due to the comparatively low actuator power density, electromechanical devices tend to be used for narrowly localized treatments, as the electromechanical pressure wave emitters being used (electromagnetic coil actuator or piezo actuator) only achieve a shock wave-like pressure increase at one focal point, i.e. initially only pressure waves are present on the path way to the focal point, but no shock waves. Such devices are nevertheless subsumed under the common generic term “shock wave generators” or “shock wave devices”.

There are also ballistic pressure wave generators, which have a tube in the therapy head in which an electromagnetically or pneumatically accelerated projectile is placed at the end of the tube on a part of the body and the pressure waves created by the impact of the projectile spread radially from the point of impact. In addition to shock wave generators, there are also devices that emit ultrasonic waves or bursts. These waveforms have equally high positive and negative pressure components. The acoustic energy that is introduced into the tissue in this way can therefore only be attributed in equal proportions to positive and negative pressure amplitudes and therefore has a different quality to the acoustic energy of an electrohydraulic shock wave generator, which is primarily emitted via positive pressure wave amplitudes. Due to the continuous, sinusoidal signals and the danger of negative pressure amplitudes, caution is required when using ultrasonic transducers if they are operated at high power. In this respect, the acoustic energy alone is not a sufficient measure for assessing the safety of treatment with shock wave generators, as the level of the local pressure amplitudes and the ratio of positive and negative pressure amplitudes are also important. In addition, the local energy is not a relevant measure, particularly in the case of focusing, as the local intensity (energy/per area) is decisive.

What all devices for generating shock or pressure waves usually have in common is that they have applicators that are connected to the shock wave generator via a current-carrying high-voltage cable (and possibly also with fluid lines). Part of the applicator is the therapy head, via which the shock or pressure waves can be applied to the body or the patient's head.

CN105126262B discloses a method and apparatus for ultrasound-based activation of cells. The method includes the treatment of pathological conditions including but not limited to Parkinson's disease, Alzheimer's disease, stupor, epilepsy, stroke, melancholia, schizophrenia, addiction, nervous system pain, cognitive/memory disorders, diabetes, obesity, obsessive-compulsive disorder, traumatic brain injury, post-traumatic stress disorder (PTSD), spinal cord injury, peripheral nervous system, migraine, epilepsy disease of human or animal body.

Pathological protein deposits exhibit specific physical properties that lead to a distinct dynamic behavior. The physical parameters are used to determine specific altering mechanical fields that lead to shock wave-induced fragmentation and dissolution of pathological protein deposits.

The nerve cells of the human brain communicate with each other via electrical impulses. In order for the neighboring cell to receive the information, the electrical impulses are converted into chemical messengers.

Amyloid plaques are hard, insoluble protein deposits made up of the protein fragment “beta-amyloid”. This protein fragment is no longer broken down by the brain in Alzheimer's disease.

The second type of protein deposits are the so-called tau fibrils. These are insoluble, twisted fibers that are composed of the tau protein. This protein is an important component of transport structures within nerve cells. In Alzheimer's patients, tau proteins hinder the transport of nutrients.

In many Alzheimer's sufferers, amyloid is not only found between the nerve cells, but also in smaller blood vessels in the brain. This is why around 15 percent of all Alzheimer's suffer not only from Alzheimer's-type dementia, but also from vascular dementia. The small blood vessels in the brain are constricted with amyloid, which in turn can trigger strokes.

Plaques that occur in Alzheimer's disease produce free radicals and oxygen-containing compounds.

Beta-amyloid refers to proteins that are considered to be the main triggers of Alzheimer's and other dementia-related changes. These proteins are also found in healthy people. They are pathogenic if they cannot be broken down. They then lead to a disruption of neuronal impulses and signals cannot be passed on.

The plaques are highly variable in shape and size; in tissue sections immunostained for AD, they comprise a size distribution curve with an average plaque area of 400-450 μm².

Amyloid materials are very stiff. The physical properties of amyloid plaques vary, e.g. the modulus of elasticity varies between 108 Pa and 1010 Pa. Beta-amyloid is a fragment of a protein that is cut out of a larger protein called APP (amyloid precursor protein). In the healthy brain, these fragments are broken down and destroyed. In Alzheimer's disease, however, they accumulate to form hard, insoluble plaques.

AU2003250102B2 describes a method to develop a vaccine against Alzheimer's disease using beta-amyloid fragments. Using an elastography imaging modality, the mechanical properties of the protein deposits are determined.

In JP2020501734A, ultrasonic energy is used to treat degenerative dementia. The focus of the ultrasonic transducer beam is directed to a target area of the brain, which helps to remove material that accumulates in the interstitial pathway that is at least partially responsive to degenerative dementia. In one example, the target area of the brain may include the hippocampus and the degenerative dementia may be Alzheimer's disease. Ultrasound beams can stimulate brain tissue at a frequency that matches the naturally occurring deep sleep burst frequency of neurons and subsequent astrocytic activation patterns, driving convective processes in response to solute release in the brain. For example, a transducer can generate a burst frequency of 1 to 4 hertz to stimulate deep sleep brain function that helps remove amyloid plaque.

In EP2722012B1 a device for treating the human or animal brain with shock waves by means of a shock wave transducer is described, which makes it possible to apply a predetermined dose to a treated area and/or volume of brain tissue which is significantly larger than the size of the focal spot of the shock wave transducer. An energy of 0.01 to 1 mJ/mm², most preferably 0.1 to 0.3 mJ/mm², is suggested after absorption correction. The pulse rate is between 1 and 20, most preferably 3-8 pulses per second. There must be a minimum dose to achieve a therapeutic effect, e.g. for thrombolysis, increased blood flow, metabolism, removal of amyloid beta or stimulation of nerves or brain cells. According to EP2722012B1, exceeding a maximum dose must be avoided at all costs, as this can lead to dangerous side effects such as bleeding.

Due to the system-related focusing described above in electromechanical shock wave devices for achieving a shock wave, a “mapping device” (or 3D tracking system) was presented in the patent specification in order to map the movement of the focal spot over several positions together with the shock or pressure wave dose applied at each of the positions. With such a system, an attempt is made to check the treatment or avoid side effects on the basis of displayed patient data, including ensuring that the same areas of the brain are not irregularly exposed too frequently or too intensely due to the system-related focused wave fields.

Through very precise, monotonous repetition of shock or pressure waves with almost the same amplitude, as is sometimes the case with electromagnetic or piezoelectric shock wave generators (subsumed under “electromechanical” shock wave generators), it cannot be ruled out that locally high energy concentrations can occur in the brain.

Focused electromechanical shock wave devices can therefore lead to high tissue exposure and are presumably used for this reason with complex 3D tracking systems. The tracking systems can at most roughly determine the exposure or locally administered dose, but do not provide a detailed analysis of the effects at the tissue or cell level in the brain. However, it is precisely the precise consideration of the local physical effect of the shock waves on the skull and the tissue and cell structure in the brain that enables the treatment to be as effective and safe as possible. In addition to the density and elasticity of the tissue to be treated and the skull, the angle of incidence of the shock waves on the skull also plays a role, as the direction of the wave propagation can change considerably due to the curved skull. As a result, different angles of application of focused shock or pressure waves can lead to unintentional localized high energy concentrations in the brain.

Measurements and simulations have also shown that it is difficult to penetrate the human skull (which is denser and stiffer than the brain) with shock waves. Exposure to shock waves, which have a lot of energy in high frequency bands, can damage the tissue near the skull in the brain, among other things due to the increasing absorption or local heating with frequency.

U.S. Pat. No. 8,697,627B2 indicates that neuroinflammatory processes also contribute to the pathophysiology of Alzheimer's disease and that microglia, the resident inflammatory cells of the brain, are in a highly activated state. Extracellular plaques are clumps of a normally harmless protein called beta-amyloid (Aβ), which can impair communication between brain cells.

U.S. Pat. No. 8,697,627B2 discloses a method of reducing amyloid plaque burden using a low dose of a p38 MAPK inhibitor. The method comprises imaging the brain, determining the number of amyloid plaques, and administering a therapeutically effective amount of a p38 inhibitor when the number of amyloid plaques exceeds a predetermined threshold.

### SUMMARY

It is the object of the invention to provide a device, a system, a method and a computer program with which the disadvantages of the known are overcome, and in particular to disclose an arrangement for the extracorporeal treatment of AD and dementia in AMF and AEF and the control of the device, by which a high effectiveness and safety in the treatment is achieved, so that in particular high pressure peaks in the brain are avoided. It is also important to optimize the penetration of the skull with a shock wave.

In particular, a further object of the invention is to achieve that pathological protein deposits in the brain are fragmented and dissolved, anti-inflammatory processes are induced, blood circulation in the brain is improved and the formation of new blood vessels (angiogenesis) is initiated.

Solutions to the problem are described in the following disclosure. Various embodiments and improvements are noted in the description and claims that follow.

The problem is solved by an electrohydraulic device for extracorporeal shock wave therapy, in particular for the treatment of Alzheimer's disease, which comprises at least one shock wave generator and at least one shock wave applicator. The shock wave applicator comprises at least the actual therapy head, which can be applied to the patient, and in particular a connecting cable and a plug.

The device has a capacitor discharge resonant circuit whose discharge frequency distribution has a maximum which is below 350 kHz, preferably below 300 kHz, more preferably below 250 kHz, still more preferably below 200 kHz, still more preferably below 150 kHz, still more preferably below 100 KHz.

The discharge frequency distribution can have several local maxima. At least one of these, preferably the main maximum, is in the low-frequency range.

For given characteristic values of the discharge capacitor, for example a capacitance between 10-800 nF, in particular 150-250 nF, and/or a discharge voltage of the capacitor between 1 kV and 20 kV, preferably between 1 kV and 7.5 kV, and/or an energy stored in the capacitor of the discharge resonant circuit of 0.5 J to 25 J, in particular 0.5 J to 5 J, the inductance, in particular the length of a cable between the shock wave generator and the shock wave applicator, is designed such that the position of the maximum of the frequency distribution according to the invention is achieved.

The problem is further solved by a device for extracorporeal shock wave therapy, in particular as described above, in particular for the treatment of Alzheimer's disease, namely an electrohydraulic shock wave device comprising at least one shock wave generator and at least one shock wave applicator. The shock wave applicator comprises at least the actual therapy head, which can be applied to the patient, and in particular a connecting cable and a plug.

The device comprises a frequency control unit adapted to adjust the shock wave generator so that a capacitor discharge resonant circuit of the device has a discharge frequency distribution whose maximum is below 350 kHz, preferably below 300 kHz, more preferably below 250 kHz, still more preferably below 200 kHz, still more preferably below 150 kHz, still more preferably below 100 KHz.

Such a device is capable of emitting shock waves with frequency bands whose dominant maxima lie in the range between 80 KHz and 850 kHz, preferably between 80 KHz and 450 kHz, even more preferably between 80 kHz and 350 kHz. The frequency bands refer to the frequency distribution in the Fourier-transformed pressure curve of the shock wave that has left the applicator. The dominant maxima of the shock wave emission do not have to lie in the preferred frequency ranges, as long as there are sufficiently strong maxima in these ranges to achieve the desired treatment effect. Specifically, in the range between 80 KHz and 850 kHz, preferably between 80 KHz and 450 kHz, even more preferably between 80 KHz and 350 KHz.

The dominant maxima of the shock waves contain at least 50%, preferably 70% and even more preferably 90% of the total energy of the shock wave.

According to the invention, mechanical fields are used. The mechanically effective, time-varying fields are adapted to the areas to be treated. The mechanical fields, in which forces act locally, are caused by shock waves and propagate through the brain. The fields or shock wave fields are characterized by the shock wave pressure amplitudes, the frequency components of the shock waves, the time interval between two or more successive shock waves, the wave velocity of the shock waves (i.e. the pulse frequency or impulse rate), and the frequency of the shock waves. The wave velocity of the shock wave, which propagates at a higher speed than the speed of sound of the transmitting medium and retains its shock wave character despite absorption during propagation due to non-linearities, as well as local vibration properties of the skull and brain (in particular density, stiffness and absorption). The term “local” refers to areas in the skull or brain down to the smallest structures.

Shock wave fields are preferably used, which propagate through the skull with low loss via low-frequency bands and apply evenly distributed pressure fields in the brain, leading to selective fragmentation and dissolution of pathological protein deposits.

By applying the shock waves via low-frequency bands and simultaneously treating larger areas of the brain, pathological protein deposits in the brain are fragmented and dissolved, anti-inflammatory processes are induced, blood circulation in the brain is improved and the formation of new blood vessels is initiated.

The frequency control unit can be designed to adjust the position of the frequency of at least the maximum of at least one of the frequency bands of the pressure signal or preferably to shift it to lower frequencies. In particular, the setting can be made by setting a discharge frequency of the electrohydraulic shock wave generator.

The discharge frequency of the discharge resonant circuit of a shock wave generator according to the invention is preferably below 450 kHz, more preferably below 400 kHz, more preferably below 350 kHz, more preferably below 300 kHz, more preferably below 250 kHz, more preferably below 200 kHz, more preferably below 150 kHz, more preferably below 100 KHz.

The capacitance of the capacitor of the discharge resonant circuit of a shock wave device according to the invention can be 50-800 nF, preferably 100-400 nF, more preferably 100-300 nF, more preferably 150-250 nF.

The voltage of the capacitor of a discharge resonant circuit in a shock wave device according to the invention can be between 1 kV and 20 kV, in particular between 1 kV and 10 kV, further in particular between 1 kV and 7.5 kV.

The discharge resonant circuit of the device can have a capacitor and the energy stored in the capacitor of a shock wave device according to the invention is 0.5 J to 25 J, in particular 0.5 J to 5 J.

The inductance of the discharge resonant circuit of the electrohydraulic shock wave device can be increased by an additional inductance and thus the discharge frequency can be significantly reduced, whereby more energy of the shock wave propagates in low-frequency frequency bands and can thus also penetrate the skull more easily, because the higher density and strength of the skull acts like a low-pass filter, which preferentially transmits pressure waves below about 450 kHz and very strongly attenuates or dampens high-frequency pressure waves from propagating into the brain.

With electrohydraulic shock wave devices, the discharge frequency of the capacitor can be reduced, for example, by introducing an additional inductance.

The frequency control unit can be designed to adjust the discharge frequency distribution by changing the inductance of the discharge circuit, in particular by determining the length of a cable between the shock wave generator and the shock wave applicator.

A coaxial high-voltage cable, which connects the shock wave generator to the applicator or therapy head, has an inductance of approx. 278 nH/m, for example. If the coaxial high-voltage cable is 1.5 m long, for example, it has an inductance of approx. 417 nH. In order to significantly reduce the discharge frequency, this inductance of the discharge circuit must be massively increased, e.g. to about twice, preferably about three times, even more preferably about four times, even more preferably about five times this inductance or even more preferably to a value that is more than five times the inductance.

The additional inductances can be realized, for example, by using long coaxial high-voltage cables between the shock wave generator, in particular its high-voltage module, and the shock wave applicator, whereby the coaxial high-voltage cables are longer than 1.5 m or more advantageously over 2 m to or even more advantageously over 3 m or even more advantageously over 5 m for use. The coaxial high-voltage cables can also have lengths of up to 10 meters, 15 meters and more. The coaxial high-voltage cables including the therapy head can be replaced.

The inductance can be changed, for example, by switching between coaxial high-voltage cables of different lengths or by connecting an additional coaxial high-voltage cable between the therapy head and the shock wave generator. Switching should not be carried out during operation and must be carried out in a high-voltage safe manner.

The coaxial high-voltage cables can also be partially installed in the stand-alone appliance. The length of the built-in coaxial high-voltage cable is preferably longer than 2 m, in particular longer than 5 m, and the coaxial high-voltage cable is particularly preferably between 9 and 20 meters long. In particular, the length of the cable can vary between 2 m and 20 m.

The coaxial high-voltage cable can run back and forth several times between the applicator and the plug on the shock wave device, whereby critical bends in the high-voltage cable can be accommodated in the plug or in the applicator therapy head.

Alternatively or additionally, a toroidal or toroidal core coil can also be used in the shock wave device, through which the current-carrying conductor of the coaxial cable is passed once or several times.

One or more coils with different inductances can be arranged in a cassette, in particular a shielded cassette, which can be retrofitted to an existing shock wave device.

The frequency control unit can be designed to add a coil, remove a coil from the discharge circuit or switch back and forth between coils with different inductances.

Alternatively or additionally, means known from the state of the art in electrical engineering can be used to achieve a lower discharge frequency of a capacitor resonant circuit.

The inductance of the discharge resonant circuit can also be reduced to half, the third, fourth or fifth part or an even smaller value in order to increase the discharge frequencies accordingly, whereby more energy of the shock wave propagates in higher frequency bands.

This can be achieved, for example, by using high-voltage cables with low inductance, e.g. a shorter axial high-voltage cable, in particular shorter than 1.50 m, preferably shorter than approx. 1.10 m and even more preferably shorter than 1.0 m.

Basically, piezoelectric, electromagnetic and electrohydraulic shock wave generators can be used. Ballistic generators can also be used for localizations of Alzheimer's areas close to the cranial bone.

However, a non-repetitive, monotonic application of the shock waves with a certain variability from shock wave to shock wave, i.e. alternating characteristics of the shock wave field, is advantageous for the protection of the cells and neurons. A variability of the amplitudes of the pressure curves of 5% is favorable, even more favorable of 10%. The variability in the frequency of the dominant frequency bands can also be 2-5%, preferably 2-10% and more preferably 2-15%.

The problem is also solved by a device for extracorporeal shock wave therapy, in particular for the treatment of Alzheimer's disease, in particular as described above, which comprises at least one, in particular electrohydraulic, shock wave generator and at least one, in particular electrohydraulic, shock wave applicator. The shock wave applicator comprises at least the actual therapy head, which can be applied to the patient, and in particular a connecting cable and a plug.

The device comprises a pulse control unit designed to adjust the electrohydraulic shock wave generator so that the pressure characteristics of the shock wave ideally have rise times of 6-50 ns, preferably 6-22 ns, more preferably 9-12 ns. Such short rise times can preferably be realized with a priori shock waves by electrohydraulic shock wave generators.

The pulse control unit can be designed to set the rise time. A shorter or longer rise time can be achieved, for example, with a lower or higher inductance of the discharge circuit.

The rise time also depends on the discharge power of the discharge resonant circuit or the amplitude of the generated shock or pressure wave due to the non-linear steepening effect of shock or pressure waves. A shorter or longer rise time can be achieved with a higher or lower discharge power of the discharge circuit.

It has been shown that fragmentation and triggering of the amyloid deposits occurs through shock waves that steepened over a short period of time.

Tau fibrils can also be fragmented and triggered by applying shock waves that are steepened within periods of 6 to 16ns, preferably 8-10ns.

The device for extracorporeal shock wave therapy as described above, in particular an electrohydraulic shock wave device, comprises a pulse frequency control unit designed to adjust the shock wave generator so that shock waves are delivered with a pulse frequency (or pulse rate) greater than 10 Hz, preferably greater than 15 Hz, preferably with a pulse frequency greater than or equal to 20 Hz, more preferably greater than or equal to 30 Hz, more preferably greater than or equal to 40 Hz, more preferably greater than or equal to 50 Hz and most preferably in the range from 50 Hz to 100 Hz.

Preferably, the pulse frequency control unit is designed to change the discharge rate of an electrohydraulic shock wave generator. There are many technical possibilities for changing the pulse frequency and reference is made here to the broad state of the art in electrical engineering. For example, the capacitor can be charged more quickly between two pulses using a larger charging device.

The elongation speed of healthy brain areas and protein deposits plays an important role in the fragmentation and dissolution of amyloid deposits and tau fibrils. The density and stiffness of the protein deposits are significantly higher than in healthy brain areas. For this reason, the frequency spectrum of the shock waves after propagation through the skull must contain relatively high-frequency and high-level frequency bands in the range above 100 kHz, preferably above 140 kHz, even more preferably above 180 kHz, and even more preferably above 250 kHz. This is because the elongation recovery speed of the amyloid deposits and the tau fibrils is smaller than in healthy neurons. The second shock wave should be applied before the amyloid deposits have fully recovered. The second and subsequent shock waves cause further elongation of the amyloid deposits until lethal elongation and fragmentation of the harmful protein deposits are reached.

The pulse frequencies or pulse rates of the shock waves are determined by calculation and validated in tests with amyloid deposits. In general, the frequencies for Alzheimer's treatment are between 20 and 30 Hz. For some early-forming deposits, the frequency is 40 Hz. High pulse frequencies of up to 100 Hz can be achieved with electrohydraulic shock wave generators. Shock wave pulse frequencies greater than 15Hz, preferably 15 Hz to 25 Hz, even more preferably around 35 Hz to 45 Hz and also preferably between 45 Hz and 55 Hz are therefore used in therapy for the effective removal of amyloid structures. The high pulse frequency can reduce the power of the individual shock wave, but this also serves to avoid high pressure shocks and tissue damage. In addition, the time interval between each individual shock wave and the preceding shock wave and the maximum amplitude of each individual shock wave can be set. In this way, shock wave sequences of any length can be realized. In the sequences, for example, the amplitudes of the shock or pressure waves can increase and the pulse frequency can rise. If the inductances can also be changed electronically or change with the amplitude level or pulse frequency, the frequency bands in which the energy is applied may also change.

Several identical or different shock wave sequences can then be applied one after the other with the applicator or therapy head.

Specific patterns of shock waves resulting from an Al-supported analysis or from successful clinical tests can also be used. Pulses can also be targeted at a brain region from different directions using two or more applicators or therapy heads, thereby increasing the elongation of the amyloid deposits, in particular the shear elongation.

Amyloid deposits are completely fragmented and dissolved after 4-5 shock wave treatments. Treatments should be carried out daily, but not less frequently than once or twice a week. Shock wave treatments can be repeated every 4-5 days.

Between 1000 and 4000 shock waves, preferably 2000-3000 shock waves, can be applied per treatment.

The device for extracorporeal shock wave therapy may have a therapy head designed so that the device can emit weakly focused or unfocused or divergent shock or pressure waves.

Localized damage to the brain can be avoided with weakly focused, unfocused or divergent shock waves.

The device, in particular an electrohydraulic shock wave device, can comprise a focus control unit and at least one applicator, preferably with a reflector and a pair of electrodes. The focus control unit can be designed to focus and defocus the shock waves emitted. In particular, the focus control unit can be designed to shift the position of at least one electrode in the reflector or to exchange an electrode for an electrode with a different geometry (different position of the electrode gap).

Especially with electrohydraulic shock wave generators, the type of reflector, in particular its shape, can also have a very large influence on the induced shock wave field. It is well known that the shock waves are strongly focused in ellipsoidal reflectors when the discharge takes place at the focus point F1. With paraboloid reflectors, the focusing is generally much weaker or the focus F2 is at infinity if the discharge takes place at the paraboloid focus. The axial length of the reflectors compared to the largest diameter is also an influencing factor for focusing, as is the position of the two electrodes or the electrode gap. Axial displacement of the position of the electrode gap, e.g. in the direction of the reflector outlet, can result in slightly focused or unfocused shock wave propagation. In extreme cases, divergent shock wave propagation is also possible. Paraboloidal and conical reflectors can be used for this purpose. So-called free-form reflectors can also be used. The physically effective axial length of the reflector (inner side) is preferably 40 mm from the reflector edge, even more preferably less than or equal to 35 mm or even more preferably less than 30 mm.

Simulations have shown that a ratio of the axial reflector length to the largest reflector internal diameter (only the acoustically relevant internal part of the reflector is considered here) of approximately 1.1 to 0.9 results in a significant widening. A ratio of 0.9 to 0.8 is preferred, a ratio of 0.8 to 0.7 is even more preferred, and a ratio of 0.7 to 0.5 is even more preferred.

In addition, the edge of the reflectors can be rounded, preferably with a radius of 3 mm to 5 mm, more preferably with a radius of 6 mm to 10 mm. The rounding results in less scattering.

The focusing can also be reduced by offsetting the electrodes and thus the position of the discharge to the geometric focus point of the ellipsoid or the focus point of the paraboloid. A distance of 1 mm to 10 mm from the focus can be useful, a distance of 2 mm to 8 mm is preferred, and a distance of 3-7 mm is even more preferable.

It is generally advantageous if the distance to the geometric focus in the direction of the shock waves to be applied can be adjusted. Focusing can also be reduced by deliberately positioning the electrodes asymmetrically or making the reflector asymmetrical. It is well known that conical waveguides lead to spherical wave propagation. Cone-shaped or truncated cone-shaped reflectors are therefore also effective for weak focusing. If the discharge takes place as close as possible to the intersection of the axial cone generatrices, it is also possible to generate shock waves that essentially only have a spherical primary pressure wave.

A spherical shock wave can be favorable, as the shock waves are additionally focused by the rounded shape of the skull bone and the higher speed of sound in the skull bone. This means that only a slightly diverging shock or pressure wave emitted by the applicator or therapy head can achieve a practically planar, uniform and wide wave front (approximately half to a full reflector diameter wide) within the skull when it hits the curved skull in the brain and treat large areas of the brain gently.

The electrode position of the inner and/or outer conductor can be optimized in such a way that planar waves propagate in the treatment area, taking into account the curvature of the skull (this is achieved, for example, with ellipsoidal reflectors if the electrode gap is outside the focus F1). The electrode position of the inner and/or outer conductor can be adjusted manually or by replacing the electrode or also by an adjustment mechanism in which at least one electrode position is adjusted.

Taking into account the curvature of the skull, planar, unfocused waves can also be achieved in the treatment area with a divergent shock or pressure wave radiated radially by the applicator or therapy head.

If necessary, strongly diverging shock or pressure waves that hit the skull can also be used to achieve diverging waves in the skull that have even lower energy concentrations than the planar shock or pressure waves.

Conversely, the focusing effect due to the round shape and higher density or strength of the skull may be a further (possibly greatly underestimated) risk when applying focused shock or pressure waves.

The focus control unit can be designed to receive data on the skull, in particular on density and/or geometry, and to take this into account when focusing or adjusting the focus of the shock waves. The local skull structure can lead to stronger focusing or defocusing of the emitted shock wave.

The focus control unit can be designed to vary the amplitude so that low-focused or non-focused shock waves are emitted that exhibit variability with regard to the amplitude of the shock or pressure waves.

The ratio of the absolute value of the positive pressure to the absolute value of the negative pressure amplitude, abs (p+)/abs(p−), is greater than 1, preferably greater than 2, even more preferably greater than 3.

The amplitude variability is characterized as a deviation from the mean value of the maximum shock or pressure wave pressure amplitude. An amplitude deviation of up to 5%, preferably up to 10% and even more preferably of 11-25% is advantageous.

Rise time variability can also be characterized in a similar way. The rise time, which in the case of the shock or pressure wave is defined as the period in which the amplitude rises from 10% of the maximum value to 90% of the maximum value (p+), can deviate from an average value of the rise time. A rise time deviation of up to 10%, preferably up to 20% and even more preferably 20-50% is advantageous.

Electrohydraulic shock wave generators offer a further advantage here.

An inherent variability of the alternating amplitude of the shock waves results from the varying position of the plasma channel or gas bubble between the electrodes. This results in a further local attenuation or temporal defocusing of the shock waves in the skull over the duration of the treatment at one point when alternating shock waves are applied repeatedly. This prevents local tissue or cell structures from being damaged, as it happens much more quickly by monotonous repetition of very similar or (almost) identical pressure pulses with low dispersion (low variability or few alternating properties of the shock or pressure wave field) at the same location. The variability of the alternating pressure curves of the electrohydraulic shock wave devices is therefore a positive feature with regard to the gentlest possible treatment of brain tissue.

The invention thus enables evenly distributed pressure fields in the entire brain area and avoids pressure peaks, in particular monotonously repeated local pressure maxima, in the focal area. These advantages apply in particular to electrohydraulic shock wave generators.

An even distribution of the shock wave application can also be achieved if the device comprises two or more shock wave applicators or therapy heads that can emit shock waves simultaneously. In particular, the device can comprise two or more discharge capacitors and two or more shock wave applicators or therapy heads, each with a pair of electrodes, whereby in particular all pairs of electrodes can be ignited simultaneously or almost simultaneously by the discharge capacitors.

If necessary, only one discharging capacitor can be used (especially if the capacitor has a discharging circuit).

In particular, the individual pairs of electrodes can be ignited at different times and different sequences of shock or pressure waves can be generated.

The shock waves of the shock wave applicators or therapy heads can be ignited successively so that, for example, shock or pressure waves arrive at the treatment area from different directions in quick succession.

The device for extracorporeal shock wave therapy may comprise a control arrangement, wherein the control arrangement is designed to adopt an operating mode for the treatment of Alzheimer's disease and to specify in this mode predetermined setting parameters for the frequency control unit, the pulse control unit and/or the pulse frequency control unit.

The device can also be used for other tissues and indications if required, but can also be set up again for the treatment of Alzheimer's disease.

The problem is further solved by a system for the treatment of Alzheimer's disease comprising an extracorporeal shock wave therapy device as described above. The system further comprises a processor unit which is designed to receive diagnostic data from a data memory or a diagnostic device, to determine setting parameters for the device for extracorporeal shock wave therapy as a function of the diagnostic data, to output the setting parameters to the device for extracorporeal shock wave therapy.

The diagnostic device can be an imaging device (with a corresponding processor unit) or a device for analyzing blood values with regard to amyloid.

The system can include a diagnostic device and/or a data memory.

A so-called Precivity AD blood test can detect amyloid and be used to assess the success of treatment.

The shock wave treatments can be controlled on the basis of measurements of blood values with regard to amyloid.

If no more amyloid is detected in the blood test, treatment can be stopped. If less or more amyloid is detected than in a previous diagnosis, the shock wave treatment parameters can be adjusted accordingly.

The diagnostic data can include data on the density and/or geometry of the skull. Furthermore, other determined or calculated local physical parameters of the brain tissue such as orientation or anisotropies, structure, strength, vibration and damping behavior, cell type, cell shapes can also be used to detect diseased cell areas of the brain or to monitor the success of treatment.

The locally variable cranial curvature, thickness, density and strength can be taken into account when planning the desired shock wave characteristics.

In the case of treatment of other body parts, the diagnostic data may include data on the local density, strength, structure, vibration and damping behavior and/or geometry of tissue, organs and bones.

Taking into account the local curvature of the skull, planar, unfocused pressure waves can be generated in the treatment area using a divergent or radially emitted shock wave from the shock wave applicator, for example. In extreme cases, divergent shock waves can also be generated in the skull.

For example, the control device can be designed to adjust the electrode positions of an electrohydraulic shock wave device in such a way that planar waves propagate in the treatment area, taking into account the curvature of the skull.

The setting parameters are selected in particular from the group:
Number, position and/or alignment of the shock wave applicators or therapy heads,
Discharge frequency of the discharge resonant circuit,
Rise time of the shock wave,
Power level or charging voltage of the capacitor,
Degree of focus,
Pulse frequency (or pulse rate),
Number of shock waves to be applied,
Sequences of the individual shock waves,
Repetition rate of the sequences.

High-resolution patient data (CRT, MRI, etc.) can preferably be read in

(e.g. in DICOM) to determine setting parameters.

In this context, high-resolution can mean resolution down to cell level.

The patient data can be prepared for an FEM (finite element analysis) calculation (e.g. with MATLAB) and at least one FEM calculation (e.g. with ANSYS) can be carried out to simulate the influence of the shock waves and/or shock wave sequences at cell level.

This procedure can be used to derive setting parameters for effective and tissue-friendly treatment.

The system can include a data memory for storing patient data, calculation data and treatment parameters.

The control arrangement can be designed to store the treatment parameters determined and/or to read out the optimum local treatment parameters for a patient during treatment.

In order to apply patient-specific shock waves in the AD area, for example, CT/MRI patient data (e.g. obtained with CRT or MRI devices) can be read into a DICOM/MATLAB/FEM program system, converted into an FEM calculation model (ANSYS, PZFLEX) via MATLAB and the shock wave propagation from the applicator (possibly also at different power levels, directions, spectral energy distribution) through the skull into the brain can be calculated or solved numerically.

In addition to the density, wave velocities and absorption coefficients of the skull and brain, local structural properties in the brain are also taken into account in detail during the simulation. The results of the simulation of shock wave propagation determine the treatment parameters, such as the direction or area of treatment, the level of power to be set or the resulting pressure amplitudes, the frequency and timing or sequences of the individual pulses as well as the low-frequency frequency bands over which the energy is preferably transmitted.

The local vibration behavior of the skull bone can also be taken into account, which results from the thickness of the skull bone, its shape and its local density and stiffness values.

In addition, other patient-specific parameters or diagnostic data such as gender, age, geometric values (brain size, volume, density, skull thickness as well as skull density and skull strength), high-resolution scans of conspicuous brain areas, as well as the local effect caused in the brain can be taken into account when calculating the wave propagation when deriving treatment parameters.

By using artificial intelligence (AI) or deep learning, the most suitable parameters for shock wave treatments for AD can be calculated, e.g. on the basis of patient-specific data (CT, MRI, etc.) before and after one or more treatments, on the basis of simulation calculations, possibly on the basis of measurements (e.g. blood measurements), as well as on the basis of established treatment courses and successes.

Artificial intelligence can be used to evaluate simulation results and CT/MRI patient data before and after one or more treatments of a large number of patients.

Artificial intelligence can be used to select or assign treatment parameters to patient-specific data. However, the treatment parameters determined should always be checked by a medical doctor before treatment.

The diagnostic device can be an imaging device for 3D representations of the head and the processor unit can be designed to simulate propagations of shock waves in a head based on the image data, e.g. on the basis of CT/MRI patient data, and to determine optimized setting parameters for the shock wave treatment depending on the simulation results.

In the context of this application, CT and MRI are listed as representative of all applicable 2D and/or 3D imaging procedures.

DICOM is a current international standard that is used to store medical images such as CT and MRI scans and is also representative of all other comparable standards in this area of medicine.

MATLAB is software for solving mathematical problems and graphically displaying results and is representative of all other calculation software applications.

FEM stands for finite element analysis and is a simulation method in which the areas of a calculation area (in this context, the skull with the brain) are divided into small elements (the finite elements) in order to predict the physical behavior in the relevant (sub)areas, here in the context of the application of shock or pressure waves. The FEM is also representative of all other numerical or analytical calculation methods that are or will be relevant in this context. As an alternative to the actual FEM software, finite difference calculation methods can also be used, for example. ANSYS, PZFLEX etc. are exemplary programs for FEM calculations.

FEM simulation models are particularly suitable for the mathematical description of the complex processes of shock wave propagation. Based on the results, it is possible to “predict” the propagation of the pressure wave in the tissue and the effect on amyloid structures. This patient-specific procedure is based on the consideration of the individual anatomical structures, microstructures such as filaments and microtubules and the physical-acoustic impedance laws. By using FEM simulations, the greatest possible safety is achieved from the patient's point of view; in particular, cell and tissue damage is effectively avoided by the weakly focused or unfocused shock waves.

The term DICOM/MATLAB/FEM program system is used in this application to refer to all program systems that read in patient data, convert it into a calculation model and evaluate it numerically using software.

DICOM/MATLAB/FEM simulation model refers to the corresponding simulation model of a DICOM/MATLAB/FEM program system, which is also representative of general simulation models of comparable program systems.

The system can have a localisation system with which the at least one shock wave applicator or its therapy head is aligned with the amyloid areas. Alignment can be performed manually or by an automatic positioning device.

The localisation system can comprise a patient rigid body, an optical positioning controller, a rigid body applicator and a DICOM-MATLAB-FEM interface for shock wave propagation. Rigid body refers to a rigid body representation of the patient (or parts thereof) or the applicator, etc. For this purpose, corresponding markers are applied to the patient's head and the therapy head, which can be used to determine the exact position and alignment of the patient's head and the therapy head.

Most aspects of the invention do not only relate to the treatment of brain tissue. In principle, other areas of the body can also be treated in a similar way. For example, intestinal cells are similar to brain cells in some aspects. Gentle application also makes sense in this and other areas of the body. Thanks to the weak or unfocused shock waves, even large areas of the body can be treated evenly. Animal brains or body areas can also be treated.

The system may comprise an ultrasound device and a combination control arrangement designed to apply ultrasound waves and shock waves to the same treatment area at the same time and, in particular, to coordinate the setting parameters of the ultrasound and the shock waves.

The amyloid-destructive potential of the shockwave device can be enhanced by the shockwave-induced activation of ultrasound sensitizers, namely sonosensitizers that are activated by ultrasound (see also: Lin et al, Ultrasound Activated Sensitizers and Applications, 2019, Angew. Chem. Int. ed. 10.1002/anie. 201906823), can be improved. In particular, cavitation and thermal energy are used for triggering. In particular, shock wave-specific ultrasonic pulses (or shock waves that have ultrasonic components) can be realized. In principle, shock waves and ultrasonic waves can also be used simultaneously to trigger the ultrasonic sensitizers.

The system can comprise a medicine delivery device. The processor unit may be designed to determine dispensing parameters for the medicine delivery device depending on the diagnostic data and to transfer the dispensing parameters to the medicine delivery device.

Alternatively, the medicine delivery device can also be designed only to display the optimum medicine delivery in the form of the delivery parameters in order to then administer the medication manually.

Alzheimer's treatment by shock wave can be planned as a combination therapy with monoclonal antibodies such as the new active ingredient aducanumab or Aduhelm (USA, approved since 2021).

The system can comprise a temperature control device, in particular a cooling device, for the patient's head and the processor unit can be designed to determine temperature control parameters for the temperature control device as a function of the diagnostic data and to output the temperature control parameters to the temperature control device.

Any heat generated by the absorption of acoustic waves during treatment can be dissipated via the cooling device. In this way, the therapy can be carried out gently, and in particular without unintentional heating of the areas to be treated as well as the areas not to be treated or neighboring areas.

The problem is further solved by a method for providing setting parameters for a treatment of Alzheimer's disease with a system as described above. The method comprises the steps of providing diagnostic data from a data memory and/or a diagnostic device, and determining setting parameters for the device for extracorporeal shock wave therapy as a function of the diagnostic data.

The diagnostic device is in particular an imaging device or a device for analyzing blood values with regard to amyloid.

The method also comprises in particular the step of determining setting parameters for an ultrasound device (in particular shock wave generator and applicator with therapy head), determining delivery parameters for a medicine delivery device and/or determining cooling parameters for a cooling device.

The problem is further solved by a computer program with program code for carrying out the steps of the above method when the program is executed on a processor unit of a system as described above.

The problem is further solved by a computer program product which can be loaded directly into an internal memory of a digital computer and which comprises software code sections which perform the method steps of the above method when the program is executed on the digital computer of a system as described above.

Patient-specific simulation analyses based on CRT, MRI etc. enable evenly distributed pressure fields in the AD area. Additional localization is not absolutely necessary. Nevertheless, the control of the device is designed in such a way that the applicators flexibly integrated in the holder can be aligned to the AD area with the aid of an optical localization system and the shock waves are applied with the applicator or therapy head in a targeted manner with calculated parameters.

When used correctly, the shock wave treatment is gentle on the tissue and can be repeated until all protein deposits are dissolved and anti-inflammatory and regenerative processes are initiated.

The required total energy applied and all other treatment parameters such as energy flux density, frequency, number and sequence of shock waves can be determined by calculation and validated experimentally.

Cell mechanics can be determined both by the cytoskeletal architecture of the cell and by the mechanics of the largest organelle in the cell, the nucleus.

The information in the scientific literature on the stiffness of protein deposits varies widely. For this reason, individual AFM (Atomic Force Microscopy) measurements of protein deposits and elastographic measurements should be used. They enable the determination of patient-specific treatment parameters and serve patient safety. However, with sufficiently accurate data, typical mechanical parameters of the protein deposits can also be determined and stored and then assigned to areas in the patient's brain using the available CRT/MRI data. This speeds up the determination of treatment parameters.

In AD treatment, the focus can be placed on destroying the amyloid structures. Another approach is to use relatively low pressure amplitudes or intensities or energy flux densities (EFD) and an unfocused or weakly focused applicator to effectively treat existing inflammation in the brain, thereby reducing or even eliminating the inflammation.

Treatment with weakly focused or unfocused applicators does not require complex localization due to the lower intensities in the treatment area. Localization becomes even more superfluous if the power level is also reduced, i.e. the capacitor voltage is reduced or a high pulse frequency (or pulse rate), e.g. in the range of 20-100 Hz, is used.

### BRIEF DESCRIPTION OF THE DRAWINGS

Further information and several advantageous embodiments of the invention are illustrated below by means of illustrations/figures. Functionally identical elements are generally provided with the same reference signs.

Shown are:

FIG. 1 is a schematic representation of a first example of a device according to the invention;

FIG. 2 is a schematic representation of a second example of a device according to the invention;

FIG. 3 is a schematic representation of the therapy head of an applicator of an electrohydraulic shock wave device; applicator;

FIG. 4 is a representations of an electromagnetic therapy head of an

FIG. 5 is an illustration of another therapy head of an applicator therapy head and pressure curves;

FIG. 6 is a first example of a frequency spectrum of a pressure curve;

FIG. 7 shows a second example of a frequency spectrum of a pressure curve;

FIG. 8 is a schematic representation of a third example of a device according to the invention in the form of a ring-shaped structure with mounted therapy heads of several applicators;

FIG. 9 is a schematic representation of a fourth example of a device according to the invention in the form of a ring-shaped structure with mounted therapy heads of several applicators;

FIGS. 10A and 10B are cellular structures before and after the application of a shock wave;

FIG. 11 is a representation of the result of an FEM simulation of the application of a shock wave to cellular structures;

FIG. 12 shows cellular structures of pathological protein deposits in AD areas;

FIG. 13 is a representation of strain fields in cellular AD structures;

FIGS. 14A and 14B are representations of FEM simulation analyses of the application of shock waves to AD structures;

FIG. 15 is a fifth example of a system according to the invention;

FIG. 16 is a first example of the application of a shock wave to an area of the head;

FIGS. 17A and 17B are further examples of the application of shock waves to an area;

FIGS. 18A and 18B are further examples of the application of shock waves to a head;

FIGS. 19A and 19B show further examples of the application of shock waves to a head;

FIGS. 20A and 20B show a pressure curve of a shock wave and the associated frequency spectrum;

FIG. 21 shows an exemplary equivalent circuit diagram of the discharge resonant circuit of a shock wave generator

FIG. 22 shows an exemplary illustration of the relationship between capacitor discharge frequencies and the length of the coaxial high-voltage cable;

FIGS. 23A-23G show examples of shock wave devices with measures for changing the inductances of the discharge circuit;

FIGS. 24A and 24B show reflectors with electrodes in two different positions;

FIGS. 25A and 25B show schematic focusing effects when shock waves are applied to a head; and

FIG. 26 shows schematic steps of a method according to the invention.

### DETAILED DESCRIPTION

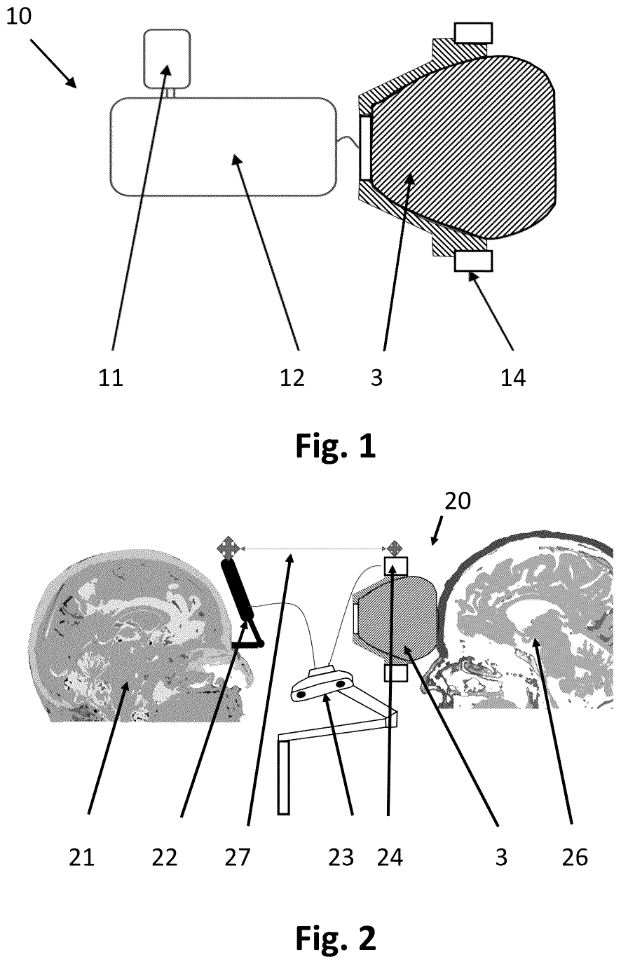

FIG. 1 shows a schematic representation of a first example of a device 10 according to the invention. The typical components of the device for the extracorporeal, tissue-gentle treatment of Alzheimer's disease with the aid of alternating mechanical fields are shown. The control device 11 of the capacitive discharge, the shock wave generator 12, shock wave applicators 3 with transmission medium and a positioning mechanism 14 can be seen. The localisation mechanism 14 ensures that the focused, low-focus, planar, spherical or divergent shock waves are applied to the relevant areas of the brain. For this purpose, the DICOM-MATLAB FEM program system is used, which determines the exact treatment parameters, such as the level of the positive and negative shock wave amplitude as well as the repetition frequency and, if necessary, the number of shock waves, through detailed analysis that takes into account the local cell properties and behavior.

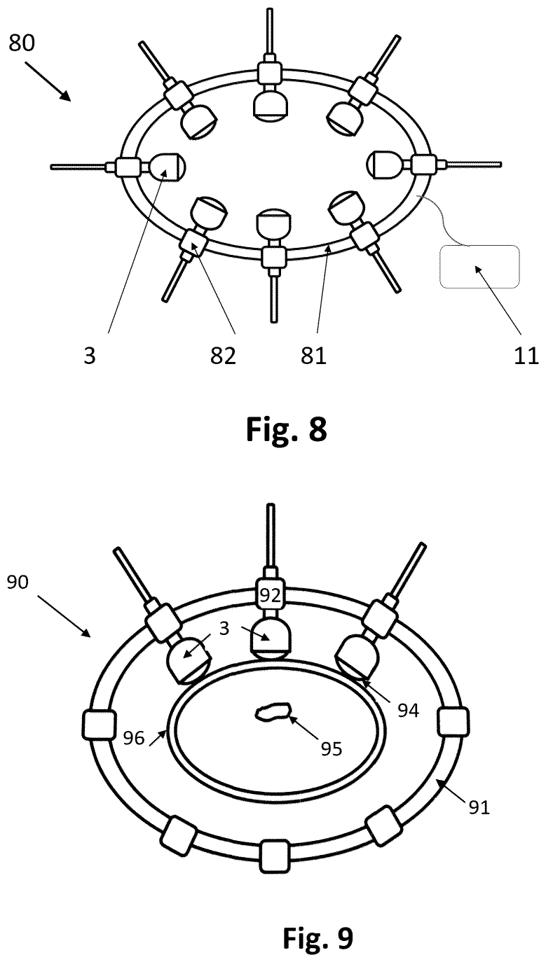

With several applicators (see FIGS. 8, 9), it is possible to superimpose shock waves from different directions, which means that the amyloid structures, which are usually also pronounced locally in different directions, can be treated effectively.

The control device 11 may comprise a frequency control unit, a pulse control unit, a pulse frequency control unit, a focus control unit and/or a control arrangement, wherein the control arrangement is adapted to adopt an operating mode for the treatment of Alzheimer's disease.

FIG. 2 shows a schematic illustration of a second example of a device 20 according to the invention, wherein a localization of the device is shown schematically.

This comprises patient DICOM data 21, rigid body model 22 of the patient's head, an optical localization 23 (e.g. an NDI Polaris system), rigid body SW applicator 24, i.e. a model of a therapy head of an applicator and its position and orientation in space, a shock wave applicator 3, DICOM-MATLAB-FEM simulation model 26 of the patient's skull, i.e. a simulation model of the head based on the measured data, which is brought into agreement with the position of the patient's head, the SW propagation into the AD area, DICOM-MATLAB-TABLEIN-FEM transfer 27 for positioning and checking the position of the therapy head on the patient and the exact alignment to the AD areas to be treated.

A patient's head is represented on the basis of patient data and its position and orientation in space, with the patient wearing glasses with markers, for example, which are detected by the optical localization system.

A rigid body model 22 of the patient's head is created from the patient DICOM data 21. A DICOM/MATLAB/FEM simulation model 26 of the skull is then created, in which a rigid body model shock wave applicator 24 applies shock waves to the skull and calculates the shock wave propagation into the AD area. The DICOM/MATLAB/FEM simulation is then used to determine the treatment parameters and setting parameters.

As described above, DICOM, MATLAB and FEM are representative components of the calculation, which are representative of similar systems that (also in combination with some of the components mentioned) can perform approximately the same or analogous simulations or calculations. Such systems are subsumed as DICOM/MATLAB/FEM program systems.

The patient puts on glasses with markers, which are used by the positioning system to record the position and alignment of the patient's head. A marker is also applied to the applicator or therapy head so that the positioning system records or calculates the exact position and alignment of the therapy head in relation to the patient's head.

The shock waves can then be applied to the patient using optical localization 23 (e.g. NDI Polaris system) to correctly position the position and direction of the real shock wave applicator 3 on the patient's real skull (according to the superimposition with the skull model 26). The coordinates of the simulation model of the patient's head must be aligned with the coordinates of the real head.

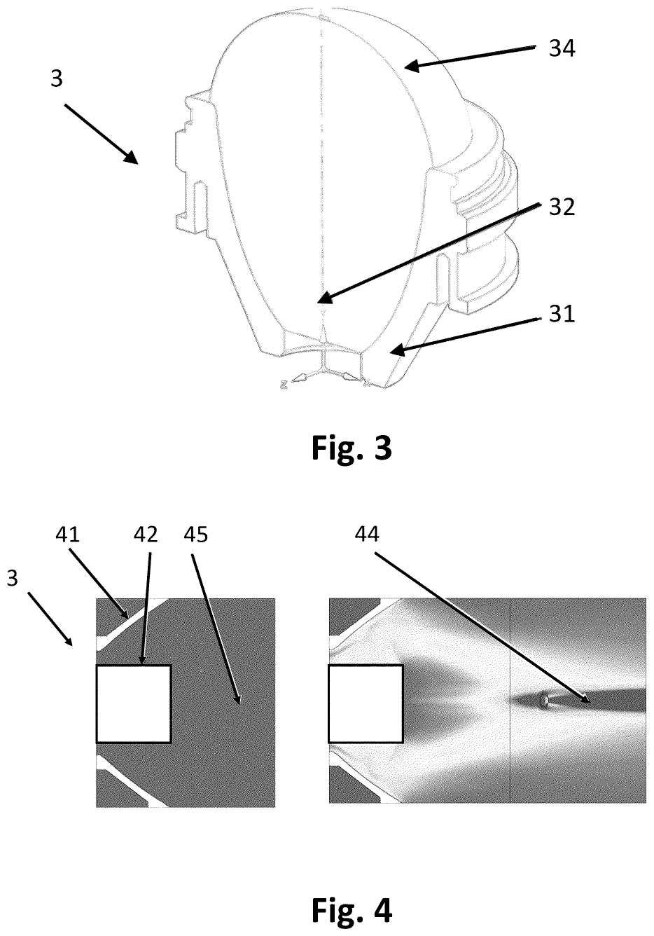

FIG. 3 contains a schematic 3D representation of an example of a focusing applicator 3 of an electrohydraulic shock wave device 10 (see FIG. 1) with the following components: Reflector 31, transmission medium 32, membrane 34.

The electrodes (not shown) are positioned and fixed in the lower part of the reflector 31 (e.g. in the shape of an ellipsoid, a paraboloid, a truncated cone or a free-form surface). The electrodes are usually positioned on the central axis. The gap between the electrodes lies, for example, in the focal point or, in the case of less focusing, also outside the focal point. Water (possibly with additives) is generally used as the transfer medium 32. During the discharge, a plasma channel is created, which leads to an explosive expansion of a gas bubble that generates the shock wave. In electrohydraulic systems, the initial shock front of the shock wave moves at a speed higher than the speed of sound of the water. The shock waves propagate in the reflector, are reflected on its inner surfaces and propagate to membrane 34, which is usually attached directly to the patient's skull with a gel (not shown in the picture).

FIG. 4 shows further illustrations of an applicator 3. The applicator 3 is part of an electromagnetic shock wave device and has the following components: Reflector 41, electromagnetic coil 42, transmission medium 4 5; The distribution of the pressure fields of the electromagnetic shock wave propagation can be seen on the right-hand side. The strong focusing 44 can be clearly seen.

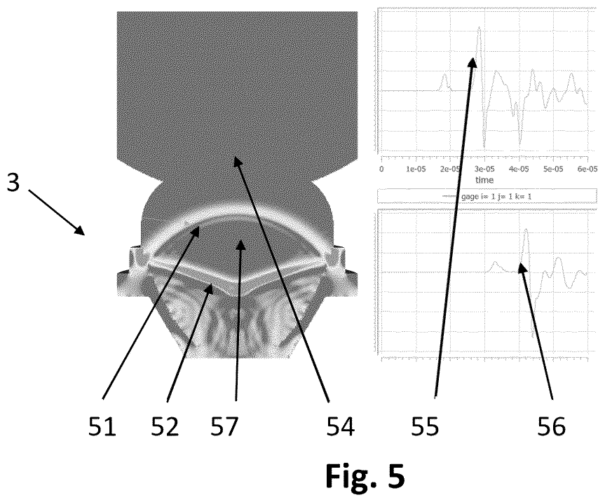

FIG. 5 shows a further illustration of an applicator 3 and associated pressure curves. FIG. 5 also shows the simulated propagation through the skullcap for an electrohydraulic shock wave generator. The primary radial pressure wave 51, the reflected pressure wave 52, evaluation point a) 57, evaluation point b) 54 can be seen. The pressure curve 55 at evaluation point a) and the pressure curve 56 at evaluation point b) are shown on the right-hand side.

The shock wave has a sharp positive peak, followed by a negative pressure component.

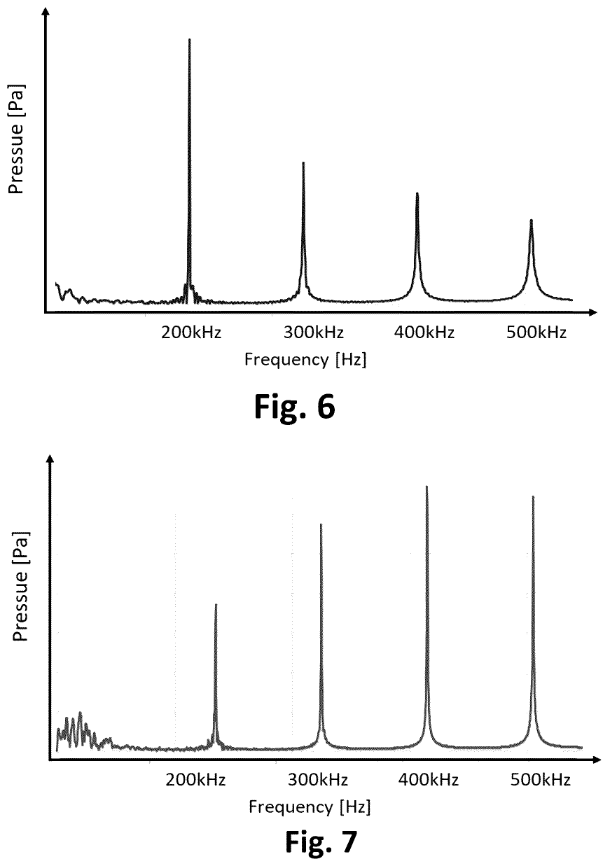

FIG. 6 shows a first example of a frequency spectrum of a pressure curve.

The example shows the distribution of the frequency bands in the FFT spectrum of a pressure curve of a shock wave at an assumed discharge frequency of the discharge resonant circuit of 200 kHz (shown as a narrowband spectrum for better understanding). It is assumed that the discharge frequency is also reflected analogously in the pressure signal. This can vary under certain circumstances, which is why this observation is only of a schematic nature. Peaks below 200 kHz are not shown here. A pronounced maximum can be recognized at approx. 200 kHz. Maxima at higher frequencies are clearly recognizable. Frequency bands of the pressure curve below 450 KHz are favorable for propagation through the skull, which acts like a low-pass filter in terms of vibration.

In principle, it is important for Alzheimer's treatment that high-frequency pressure waves reach the skull as far as possible. However, due to the described low-pass characteristics of the skull, this is only possible up to a certain frequency, e.g. 450 kHz. Pressure wave components above this frequency are not transmitted or are strongly attenuated. The lower the fundamental frequency of the pressure waves, the more high-frequency components (shown here as harmonics in the narrowband spectrum) can propagate into the skull.

FIG. 7 shows a second example of a frequency spectrum of a pressure curve.

The exemplary distribution of the frequency bands in the FFT spectrum of a pressure curve of a shock wave at an assumed discharge frequency of the discharge resonant circuit of 450 kHz is shown. Further assumptions are as described above.

A low maximum at 450 kHz and further maxima at higher frequencies can also be seen here. Here, most of the energy is transmitted in the frequency bands above 450kHz (shown here as harmonics in the narrowband spectrum). This is not optimal for penetrating the skull, as the skull has a low-pass characteristic and above 450 kHz practically no pressure waves enter the skull or are strongly attenuated.

FIG. 8 shows a schematic representation of a third example of a device 80 according to the invention. The arrangement of the device 80 with several shockwave (SW) applicators 3 is shown. The shockwave applicators 3 with transmission medium and positioning 82 of the shockwave applicators on a positioning ring 81 can be seen. Instead of a positioning ring 81, the applicators can also be mounted on a half-ring, oval or other rod-shaped positioning devices (e.g. also in spherical or hemispherical form). The shockwave applicators 3 are controlled by the control device 11, which comprises a modulation/control system. This allows the shock waves to be applied to a skull (not shown) in a targeted manner from different directions.

FIG. 9 shows a schematic representation of a fourth example of a device 90 according to the invention. The device 90 is analogous to the device 80 of FIG. 8, here only with three SW applicators 3, which are attached to the positioning ring 91 of the positioning mechanism 92 and are coupled to the skull bone 91 via transmission gel 94 to the skull 91. The SW applicators 3 apply shock waves from three directions to AD area 95.

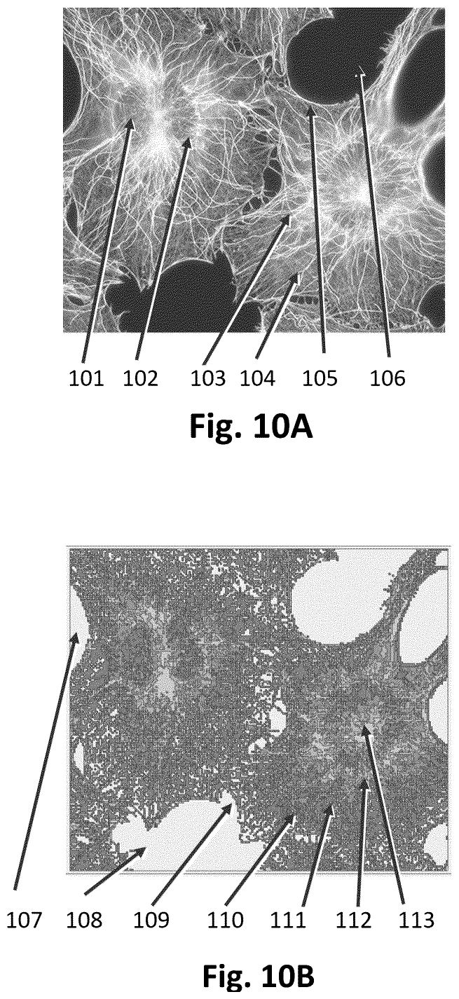

FIGS. 10A and 10B show cellular structures. FIG. 10A, 10B show the results of the FEM simulation of shock wave propagation and its effect on patient-specific cellular structures. FIG. 10A shows the nuclear membrane 102, microfilaments 103, actin filaments 104, cell membrane 105, cell nucleus 101, extracellular matrix (ECM) 106 as provided by an imaging method. FIG. 10B shows the MATLAB transfer of the patient-specific cell structures to a non-linear FEM model of shock wave propagation: 107, where ECM (extracellular matrix) 108, cell membrane 109, actin filaments 110, microfilaments 111, nuclear membrane 112 and the cell nucleus 113. A comparison of FIG. 10A and FIG. 10B clearly shows that the cellular structures are reproduced with a very high resolution in FIG. 10B in accordance with the resolution of the 3D imaging systems. Different material parameters are assigned to the structures locally. In the simulation, the shock wave penetrates the cell areas and the effect on different cell structures can be made recognizable by a color change (e.g. to represent elongation or possibly also stress in different colors).

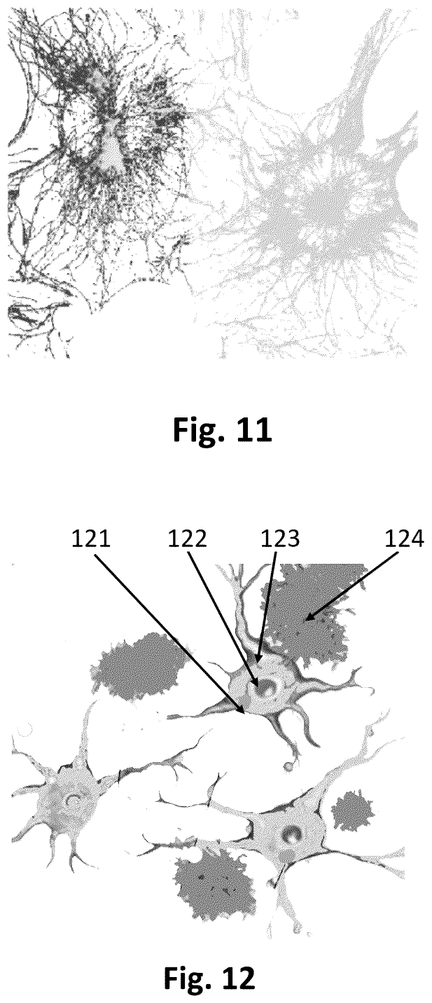

FIG. 11 shows a representation of the result of an FEM simulation. FIG. 11 shows an example of the result of the FEM simulation of shock wave propagation through a cell structure. The figure shows the strain fields at a specific point in time. You can see the shock wave front, which runs vertically in the middle of FIG. 11.

FIG. 12 shows the cellular structures of pathological protein deposits in AD areas. Tau-fibrils 121, the nucleus 122, the cytoplasm 123 and the amyloid protein deposits 124 can be seen. The dimensions and physical properties are transferred to an FEM simulation model using the programs MATLAB, PZFLEX and ANSYS and solved numerically. This results in the treatment parameters for the fragmentation and dissolution of the amyloid deposits.

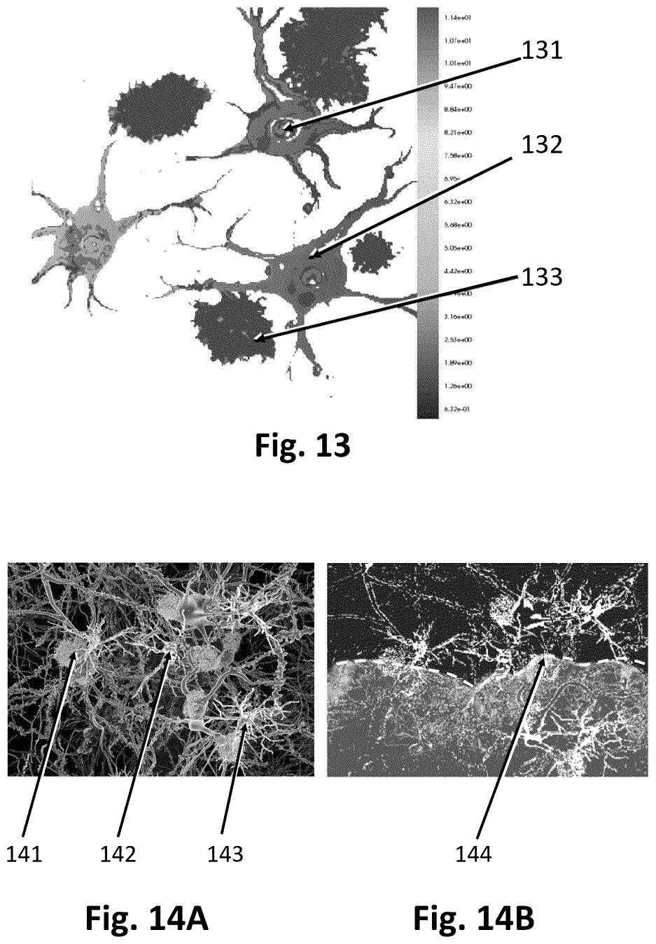

FIG. 13 shows an exemplary representation of strain fields in cellular structures. The intensity of the gray scale is a measure of the strain. FIG. 13 shows lethal strain fields in amyloid protein deposits 133. Healthy cerebral areas (nucleus 131, cytoplasm 132) remain almost unaffected, as they survive the strains undamaged due to their low stiffness.

FIGS. 14A and 14B show representations of FEM simulation analyses. FIG. 14a/b show patient-specific FEM simulation analyses of nonlinear shock wave propagation through the brain area. FIG. 14A shows the amyloid deposits 141, the neurons 142, tau fibrils 143. FIG. 14B shows the propagation of the pressure front 144 at the cellular level.

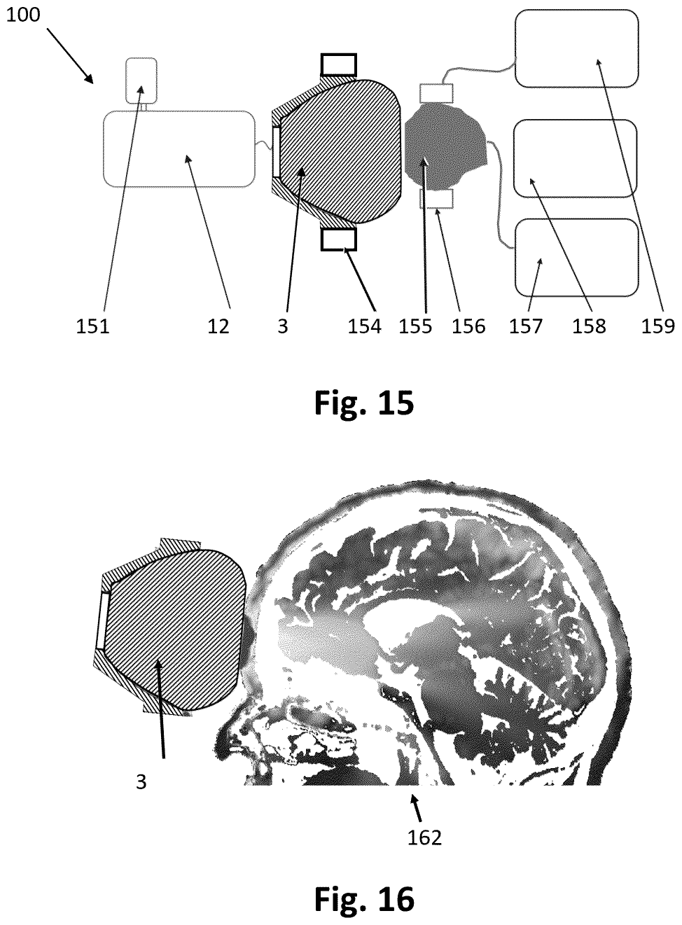

FIG. 15 shows an example system. FIG. 15 shows the components of the system 100 for the extracorporeal treatment of Alzheimer's disease using alternating mechanical fields (AMF), alternating electric fields (ETF TTF) and singlet oxygen (H3). The control device 151 of the (AMF) capacitive discharge, the shock wave generator 12, the shock wave applicators 3 with transmission medium (device with two applicators not shown), the positioning mechanism 154 of the shock wave applicator can be seen, the Alzheimer area 155 (schematic, skull not shown), the TTF electrodes 156, a temperature control device 157 for the temperature control of the Alzheimer area, the H3 supply device 158 and the TTF device 159.

FIG. 16 shows a first example of a simulated shock wave. FIG. 16 shows the representation of a simulated SW applicator 3 (with rubber membrane), transmission gel, brain section, lighter areas show the simulated pressure distribution of a shock wave propagating through the brain. It can be seen that an even pressure application is achieved over large areas of the brain with a little to unfocused shock wave.

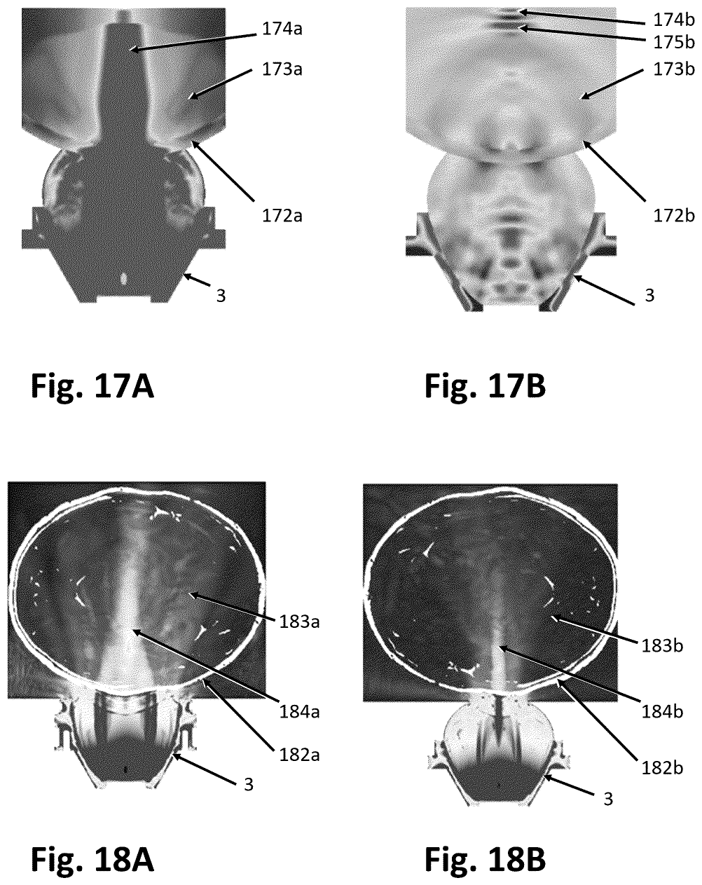

FIGS. 17A and 17B show further examples of a simulated shock wave. FIG. 17A shows the simulation of the directional effect of a weak or unfocused SW applicator 3 through a skull 172a into the brain 173a. The B treatment area 174 a (maximum values at a typical treatment pressure amplitude) is clearly visible. FIG. 17B shows a simulation of the directional effect of a weak or unfocused SW applicator 3 through a skull 172b into the brain 173b. A sequence of the temporal pressure curve of the shock wave propagation can be seen here. The first pressure wave 174b followed by a broad second pressure wave 175b with increased amplitude can be clearly seen at the upper edge of the image.

FIGS. 18A and 18B show further examples of a simulated shock wave. FIG. 18A and 18B show the difference between brain treatment with a weak or unfocused shock wave and a focused shock wave (simulations). FIG. 18A: With the weak or unfocused shock wave of the simulated SW applicator 3, a wide area of the brain 183a is treated gently with the shock wave and low pressure amplitude through the skull 182a. The treatment area 184a is shown here in light color. FIG. 18B shows that with the focused shock wave of the simulated SW applicator 3 during propagation through the skull 182b only a small area of the brain 183b is treated with the shock wave. The treatment area 184b is much smaller here than in FIG. 18A. It can be assumed that the skull 182b also has an additional focusing effect due to its curvature. Such double focusing can lead to high peak pressures and possibly to cell and tissue damage in the case of focused shock waves.

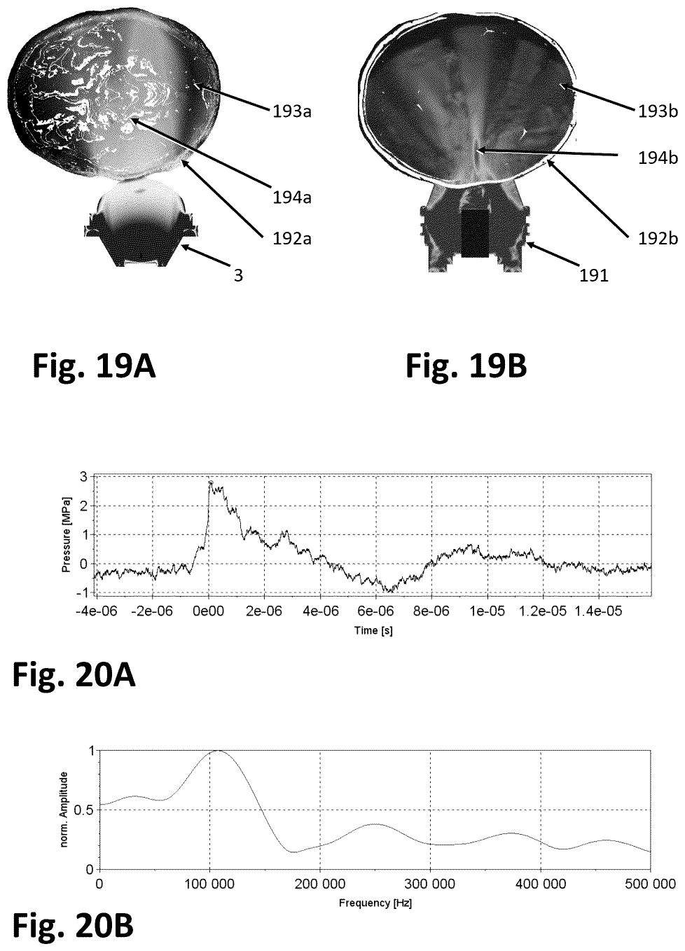

FIGS. 19A and 19B show further examples of a simulated shock wave. FIGS. 19A and 19B show the difference between brain treatment with an unfocused shock wave from an electrohydraulic SW applicator 3 and a focused shock wave from an electromagnetic SW applicator 191 in the simulation. FIG. 19A: the unfocused shock wave of the SW applicator 3 penetrates the skull 192a and a wide area 194a of the brain 193a is thus treated. FIG. 19B: the focused shock wave of the piezoelectric SW applicator 191 penetrates the skull 192b and only a very small area 194b of the brain 193b can be treated.

FIGS. 20A and 20B show a pressure curve of a shock wave and the associated frequency spectrum. FIG. 20S shows the pressure curve over time of an applicator equipped with a 10 m long applicator cable, i.e. the inductance between the electrodes and the capacitor has been significantly increased. It can be seen that the temporal increase of the shock wave is relatively slow. In FIG. 20B, the FFT analysis (normalized amplitude) of the temporal pressure curve shows several dominant frequency bands or ranges in the range from approx. 90 kHz to 450 KHz.

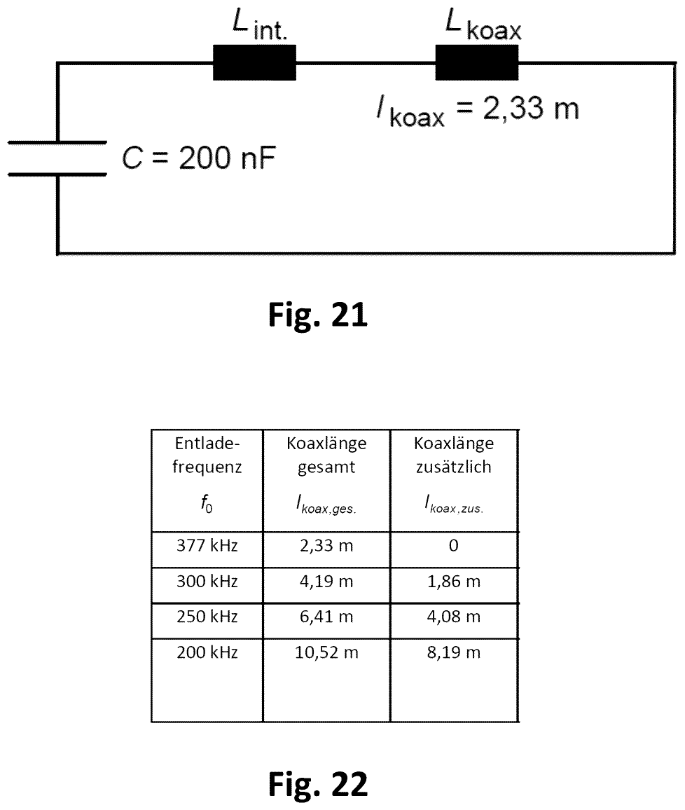

FIG. 21 shows the equivalent circuit diagram of an oscillating circuit consisting of the capacitor of the shock wave device with the internal inductance Lint and the inductance of the coaxial high-voltage cable Lₖₒₐₓ at the moment of discharge (electrodes short-circuited). The numerical values are for guidance only. For a given discharge frequency (measurement), known surge capacitance and internal inductance, the required inductance of the coaxial high-voltage cable can be calculated using the equationω=1/sqrt(C(L+Lᵢₙₜₖₒₐₓ)) and its solution for Lₖₒₐₓ. For a given length-related inductance Lₖₒₐₓ′ (typical value e.g. 278 nH/m), the length lₖₒₐₓ of the coaxial high-voltage cable can be calculated as: lₖₒₐₓ=Lₖₒₐₓ/Lₖₒₐₓ′. In order to keep the length of the coaxial high-voltage cable short, high-inductance coaxial high-voltage cables can also be used. In the state of the art, there are further possibilities for reducing or increasing the inductance of a coaxial high-voltage cable. This state of the art can also be used to increase the inductance of the coaxial high-voltage cables and enable shorter lengths of the coaxial high-voltage cable.

It should be noted that high discharge frequencies of the discharge capacitor can also be advantageous, e.g. to increase the power and generate stronger shock waves. For this purpose, the inductance of the coaxial high-voltage cable or the inductance between the surge capacitor and the applicator can be reduced. For example, the length of the coaxial high-voltage cable can be reduced or several (thinner) coaxial high-voltage cables can be connected in parallel instead of one coaxial high-voltage cable.

FIG. 22 shows an example of the relationship between the capacitor discharge frequencies and the applicator cable length. The cable lengths can be shortened by using higher length-related inductances (Lₖₒₐₓ>278 nH/m).

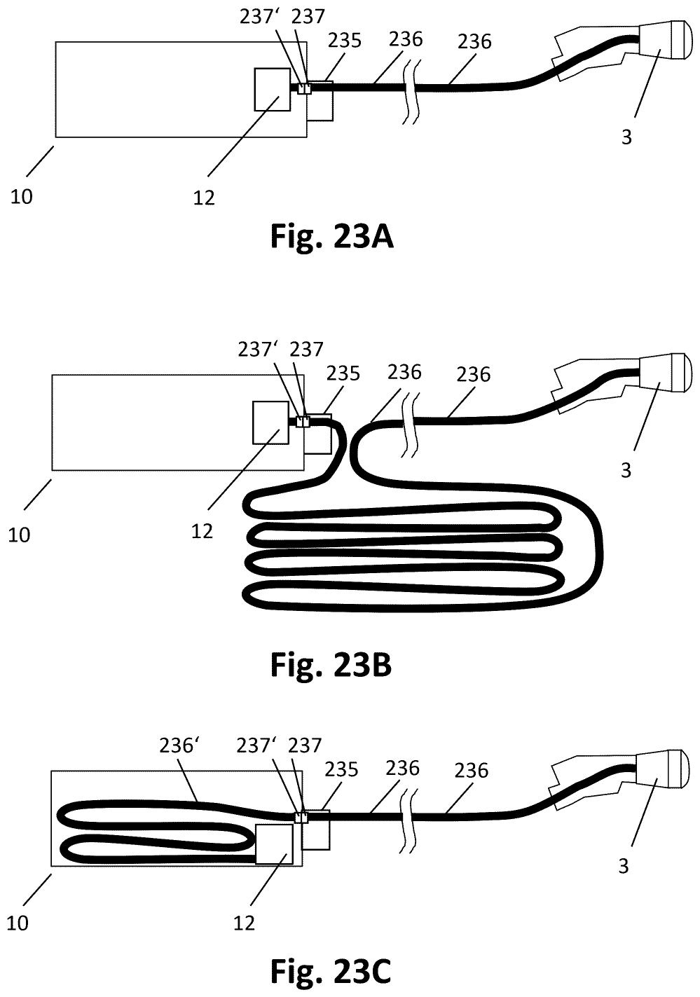

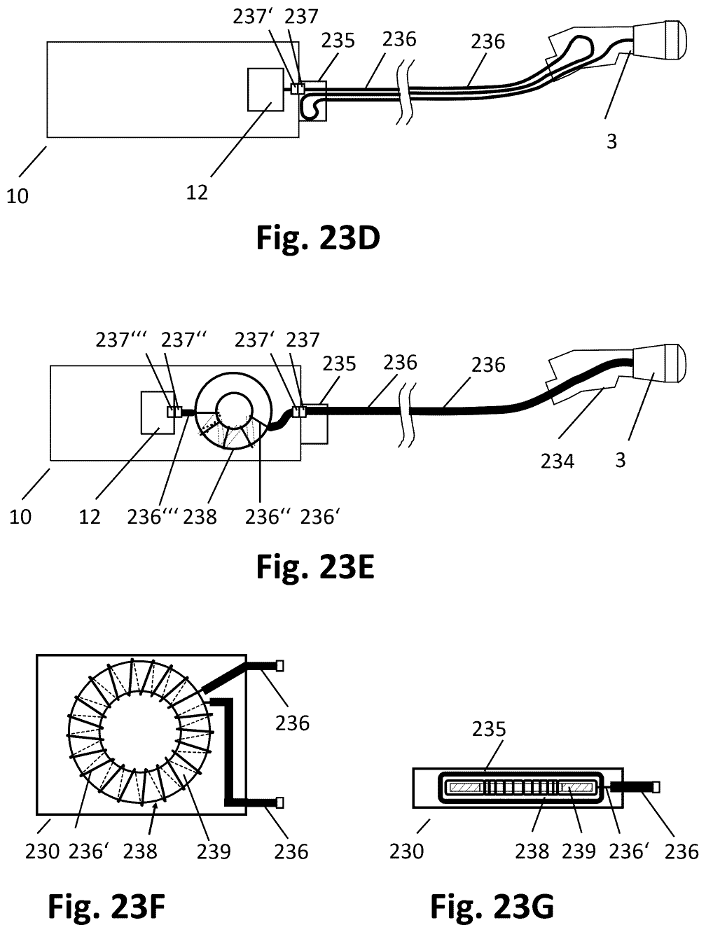

FIGS. 23A-23G show examples of shock wave devices 10 with different inductances. A shock wave device 10 with shock wave generator 12 is always shown here, for example in FIG. 23A. Only those components that are important for the explanations are shown in the figures. The applicator 3 with reflector is connected to the shock wave generator 12 via a coaxial high-voltage cable 236 and connecting elements 235, 237, 237′ (plug, socket or similar). Coaxial high-voltage cables are always used because they are electromagnetically shielded. The necessary insulation between the current-carrying inner conductor and the outer shielding results in the required relatively large bending radii and high bending stiffness of the axial high-voltage cable. The current-carrying inner conductor is not explicitly shown in the figures.

FIG. 23A shows the initial state with a short coaxial high-voltage cable 236, which is generally less than or equal to 2 m long. A much longer coaxial high-voltage cable 236 is now used as additional inductance in FIG. 23B. The length of the coaxial high-voltage cable 236 is significantly longer than 2 m, preferably longer than 5 m and even more preferably between 9 and 15 meters long. The coaxial high-voltage cable 236 can also be longer than 15 meters. A long connection between the shock wave generator 12 and the applicator 3 is definitely advantageous in many treatment situations. This means that the shock wave device 10 with the shock wave generator 12 does not have to be moved or relocated, as the applicator 3 can be used within a wide radius of the shock wave device.

In FIG. 23C, part of the coaxial high-voltage cable 236′ is accommodated in the shock wave device 10 and is then connected at the end to the shock wave generator 12. The length of the coaxial high-voltage cable 236′ in the shock wave device is more than 1 m, preferably more than 2 m, more preferably more than 5 m. When laying the coaxial high-voltage cable 236′ in the shock wave device 10, attention must also be paid to the minimum permissible bending radii of the coaxial high-voltage cable 236′. If necessary, the coaxial high-voltage cable 236′ can be subdivided several times and the pieces connected via additional connecting elements. This measure can drastically reduce the space required for the coaxial high-voltage cable 236′.

In FIG. 23D, a long, thin coaxial high-voltage cable 236 is used, which runs back and forth between the applicator 3 and the shock wave generator 12 in loops, i.e. three coaxial high-voltage cable strands then run next to each other, for example. If necessary, the coaxial high-voltage cable can also run back and forth between applicator 3 and shock wave device 10 several more times. To allow for the bending radii, the coaxial high-voltage cable 236 is bent and accommodated partly in the connector 237, 237′ or in the connecting element 235 or partly in the applicator 3, and then continues to run in the opposite direction. Instead of bending with a large bending radius, the cables can also be redirected in a space-saving manner using screw connections, e.g. in a U-shape. However, the connections must be shielded separately.

Thinner coaxial high-voltage cables can be realized, for example, by significantly reducing the voltage for electrohydraulic shock wave generation, e.g. by using particularly conductive fluids (water with additives), smaller electrode spacing or higher shock wave repetition rates.

In FIG. 23E, a toroidal or toroidal core coil is used to realize an additional inductance between the applicator 3 and the shock wave generator 12, in which the current-carrying conductor of the coaxial high-voltage cable is passed once or several times through the inside of the toroidal core coil. The inductance of the current-carrying conductor with the toroidal coil increases quadratically with the number of windings. If a hinged ferrite is used instead of or in addition to the toroidal core coil, the rise in current can be delayed until saturation current is reached, as a result of which the frequency spectrum of the surge wave pressure curve can also shift to lower frequencies.

FIG. 23F shows how a high inductance is accommodated in a small space instead of a long high-voltage cable. This is achieved by placing it in a flat cassette 230, which is part of the shock wave device 10 (see previous figures) or is mounted under or on the shock wave device. The inner conductor 236′ of the coaxial high-voltage cable 236 is laid in a spiral, resulting in a toroidal coil 238, which is arranged horizontally in the cassette 230, for example.

Depending on the size, the toroid coil can also be accommodated elsewhere in the shock wave device. The toroid coil 238 also contributes to an increase in the inductance of the installed high-voltage cable. The number of windings is quadratic and the area of the core 239 and the mean radius of the core 239 are linearly included in the inductance. With a core made of air, the number of windings can be above 30, preferably above 50 and more preferably above 100 in order to realize high inductances.

Inside the toroidal coil 238, there may be air, plastic and, in addition or instead, a ferrite or iron powder core. This can further increase the inductance or reduce the number of windings.

If a core consisting of air (or plastic) and a ferrite or iron powder core is used, the ferrite or iron powder core can be designed so that it is large enough to avoid saturation.

For electromagnetic shielding, the coil can be provided with means known from the prior art (not shown in this figure), for example a cage 235 (see FIG. 23G). Several cassettes 230 can also be used in a shock wave device 10, or several coils 238 can be used in one cassette 230.

In FIG. 23G, the cassette 230 from FIG. 23f can be seen in sectional view from the upper side. In this view, only one connection 236 can be seen (the other connection is not visible. The toroidal coil 238 can have a cage 235 as electromagnetic shielding, e.g. realizable as a wire mesh, grid, steel plate, in particular comprising magnetic materials.

The shielding may also be provided on the surface of the cassette 230 and/or the walls of the cassette 230 may comprise shielding material.

The core 239 can in turn consist of air or the aforementioned materials. In principle, a gap can also be provided in the core of the toroidal coils mentioned in the patent with a magnetically conductive annular core in order to avoid saturation of the coil.

The cassette solution enables an existing device to be upgraded or retrofitted for shock wave generation in low frequency bands. For this purpose, the cassette is connected in series via electrical lines 236 in front of the high-voltage cable of the applicator 3.

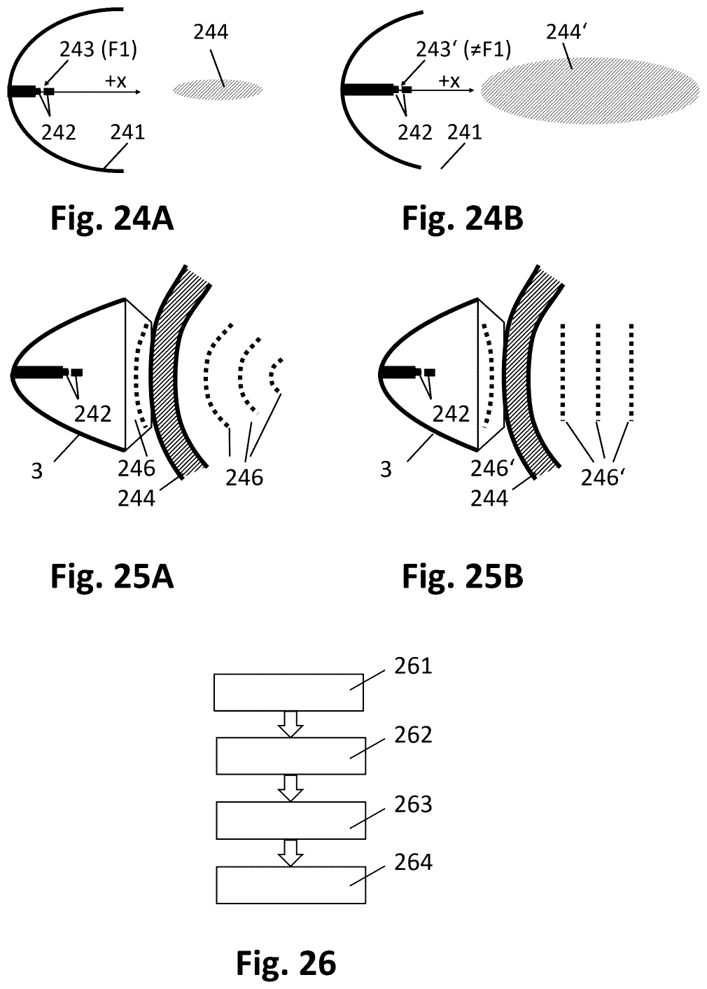

FIGS. 24A and 24B show reflectors 241 with electrodes 242 in two different positions. It is illustrated that a change in the electrode position in the +x direction or in the direction of the treatment area results in a broadening of the focus. FIG. 24A shows a reflector 241 with electrodes 242. The center of the electrodes 242 is located approximately axially in the focus point 243 of the ellipsoid or paraboloid. In FIG. 24B, the reflector 241 is axially shorter, ideally the axial length on the inside is less than or equal to 35 mm. The position of the two electrodes 242′ is ideally at a point 243′ that is not the focus point. The distance between the center of the electrode and the focal point is 3-15 mm, preferably 3-7 mm, particularly preferably 5-7 mm. This results in reduced focusing and thus a very wide and deep treatment zone with tissue-sparing pressure amplitudes is achieved. The fact that the absolute pressure amplitudes are reduced is advantageous in order to avoid overstressing or excessive heating of the brain tissue and cell damage.

FIGS. 25A, 25B show schematic focusing effects through the skull. FIGS. 25a and 25b show the influence of the focusing of the skull. In FIG. 25A, an applicator 3 emits a slightly convex shock wave. The skull 244 and its properties (higher stiffness, higher wave speed, convex shape) additionally focus the shock waves 246. This effect should definitely be taken into account during treatment. FIG. 25B shows that with a divergent emitted shock wave 246′ (dominant pressure shock) and the subsequent focusing of the skull 244 an almost planar wave propagation in the skull is achieved. The planar wave propagation in the skull prevents focusing and high, damaging pressure amplitudes. Furthermore, by taking the curvature of the skull into account, the electrode position is optimized in such a way that planar waves always propagate in the treatment area, taking the curvature of the skull into account.

FIG. 26 schematically shows steps of a method according to the invention. In particular, FIG. 26 shows the procedure for determining treatment parameters. In step 261, high-resolution (ideally down to cell level) patient data (CRT, MRI, etc.) is read in (e.g. in DICOM). In step 262, the patient data is converted for the FEM calculation (e.g. with MATLAB) and density, modulus of elasticity and other mechanical parameters are assigned. In step 263, the FEM calculation is carried out (e.g. with ANSYS) to simulate the influence of the shock waves at cell level (including repetition with changing shock wave parameters or also changed model parameters). In step 264, the treatment parameters for effective and safe treatment are derived, i.e. the setting parameters are determined. When determining the treatment parameters, an AI can also provide support by comparing previous treatment parameters with the determined treatment results for the patient currently being treated. For example, for specific regions of the patient's brain for which the fine structure has been detected, the necessary treatment parameters can already be derived using AI or by searching a database for analogies to previous treatments.

With respect to the determination of suitable parameters with AI:

The FE-Calculation and determination of suitable parameters for the shockwave treatments is done within an analysis module.

The analysis module, which can include a processor and memory, can be implemented in the shockwave device or externally on a cloud server or similar. In this case the determined suitable parameters for the shockwave treatments are transferred to the shockwave device.

The AI can also be implemented within the analysis module and/or a separate module.

The most suitable parameters for the shockwave treatments for AD are the values of the pressure amplitude, the risetime, the frequency content, the applied energy flux density, the focus, the impulse rate, the direction of the shockwave, the number of shockwaves, the sequence of shockwaves and potential further parameter, that can be controlled by the shockwave device. This applies also for the case of multiple applicators.

The training data are CAE simulations with shockwaves propagating through the tissue. The geometrical resolution of the simulations is in the range of the cell level or even higher. By permutations of the applied shockwaves or the cell model parameters, the AI is trained concerning how an effective treatment can be done, and what are the most suitable parameters.

To further enhance the determination of suitable parameters also further measurements of physiological reactions and/or biochemical changes (blood parameter) and/or patient-specific data (CT, MRI, etc.) of the patient to be treated and/or multiple other patients that have been treated can be used as additional training data (possibly in anonymized fashion).

The training data are transferred to the analysis module and analyzed by AI.

In case that a patient has to be treated, the AI provides or suggests the suitable parameters according to patient-specific conditions that are input based on the training data.

1. A device (10; 20; 80; 90) for extracorporeal shock wave therapy, comprising:
at least one shock wave generator (12);
at least one shock wave applicator (3) connected to the at least one shock wave generator; and
a capacitor discharge resonant circuit having a discharge frequency distribution that has a maximum which is below 350 KHz.

2. The device (10; 20; 80; 90) for extracorporeal shock wave therapy according to claim 1, further comprising a frequency control unit (11) configured to adjust the shock wave generator (12) so that the capacitor discharge resonant circuit has the discharge frequency distribution that has the maximum below 350 kHz.

3. The device for extracorporeal shock wave therapy according to claim 2, wherein the frequency control unit (11) is configured to adjust the discharge frequency distribution by changing an inductance of the discharge circuit, including determining a length of a cable between the shock wave generator (12) and the shock wave applicator (3).

4. The device for extracorporeal shock wave therapy according to claim 1, further comprising a coaxial high-voltage cable between the shock wave generator (12) and the shock wave applicator (3), wherein the coaxial high-voltage cable is longer than 1.5 m.

5. The device for extracorporeal shock wave therapy according to claim 4, further comprising a toroidal or ring core coil arranged between the shock wave applicator (3) and the shock wave generator (12) to realize an additional inductance, in which a current-carrying conductor of the coaxial high-voltage cable is guided once or several times through an interior of the toroidal ring core coil.

6. The device for extracorporeal shock wave therapy according to claim 1, wherein a shock wave that has left the shock wave applicator (3) has frequency bands in a range between 80 KHz and 850 kHz, and said frequency bands relate to a frequency distribution in a Fourier transform of a pressure curve of the shock wave.

7. A device for extracorporeal shock wave therapy, comprising:
at least one shock wave generator (12);
at least one shock wave applicator (2); and
a pulse control unit configured to adjust the shock wave generator (12) such that a shock wave leaving the shock wave applicator (2) has a rise time of 6-40 ns.

8. The device for extracorporeal shock wave therapy according to claim 7, wherein the pulse control unit is designed to adjust the rise time by at least one of a) changing an inductance of the discharge circuit or b) changing a setting of a discharge voltage.

9. A device for extracorporeal shock wave therapy, comprising:
at least one shock wave generator (12);
at least one shock wave applicator (2); and
a pulse frequency control unit that is configured to adjust the at least one shock wave generator (12) to deliver shock waves with a pulse frequency greater than or equal to 15 Hz.

10. The device for extracorporeal shock wave therapy according to claim 1, further comprising at least one applicator (3) with a reflector (241) and a pair of electrodes (242, 242′), and a focus control unit configured to focus and defocus delivered shock waves by shifting a position of at least one of the electrodes (242, 242′) in the reflector (241).

11. The device for extracorporeal shock wave therapy according to claim 1, wherein the at least one shock wave applicator comprises two or more shock wave applicators (3) each having a pair of electrodes (242, 242′), and at least one discharge capacitor.

12. The device for extracorporeal shock wave therapy according to claim 11, wherein the two or more shock wave applicators (3) are adapted to be at least one of a) directed at a brain region from different directions, or b) ignited in a time-delayed manner relative to one another.

13. The device for extracorporeal shock wave therapy according to claim 1, further comprising a control arrangement that is configured to adopt an operating mode for the treatment of Alzheimer's disease and in said operating mode to specify predetermined setting parameters for at least one of a frequency control unit, a pulse control unit, or a pulse frequency control unit.

14. The device for extracorporeal shock wave therapy according to claim 1, wherein the device includes auto-ignition.

15. The device for extracorporeal shock wave therapy according to claim 1, wherein at least one of:
A) the discharge resonant circuit has a capacitor with a capacitance of 50 nF-400 nF,
B) the discharge resonant circuit has a capacitor with a charging voltage of between 1 kV and 20 kV,
C) the discharge resonant circuit has a capacitor and an energy of the shock wave device stored in the capacitor is 0.5 J to 25 J,
D) the discharge resonant circuit has an inductance of at least fives times 417 nH, or
E) different coaxial high-voltage cables are switchable between the shock wave generator and the shock wave applicator.

16. A system for treating brain tissue or areas of a body, the system comprising the device (10; 20; 80; 90) for extracorporeal shock wave therapy according to claim 1, and a processor unit configured thereto,
to receive diagnostic data from at least one of a data memory or a diagnostic device,
determine setting parameters for the device (10; 20; 80; 90) for extracorporeal shock wave therapy depending on the diagnostic data, and
send the setting parameters to the device (10; 20; 80; 90) for extracorporeal shock wave therapy.

17. The system according to claim 16, wherein the setting parameters are selected from the group consisting of:
a number, position and/or orientation of the at least one shock wave applicator,
a discharge frequency of the discharge resonant circuit,
a rise time of the shock wave,
a power level or charging voltage of a capacitor of the capacitor discharge resonant circuit
a degree of focus,
a pulse frequency,
a number of shock waves to be applied,
sequences of individual shock waves, and
a repetition rate of pulse sequences.

18. The system according to claim 17, wherein the diagnostic device is an imaging device for 3D representations of a head and the processor unit is configured to simulate propagations of the shock waves in the head based on the image data and to determine optimized setting parameters as a function of the simulation.

19. The system according to claim 16, further comprising an ultrasound device and a combination control arrangement adapted to apply ultrasound and shock waves to a same treatment area.

20. The system according to claim 16, further comprising a medicine delivery device and the processor unit is further configured thereto,
to determine dispensing parameters for the medicine delivery device depending on the diagnostic data, and
to transfer the dispensing parameters to the medicine delivery device.

21. The system according to claim 16, wherein the apparatus comprises a temperature control device (157) for a patient's head and the processor unit is further configured thereto,
to determine temperature control parameters for the temperature control device (157) as a function of the diagnostic data, and
to transfer the temperature control parameters to the temperature control device (157).

22. A method for providing setting parameters for a treatment of Alzheimer's disease with the system (100) according to claim 16, the method comprising the steps of:
providing data from a data memory or a diagnostic device, and
determining setting parameters for the device (10; 20; 80; 90) for extracorporeal shock wave therapy depending on the data by using artificial intelligence to evaluate said data at least one of before or after one or more treatments,
wherein said data comprises at least one of diagnostic data, patient-specific data (e.g., CT or MRI data) before and after one or more treatments, simulation calculation data, measurement data (e.g., blood measurements) or data on established treatment courses and successes.

23. The method of claim 22, further comprising determining setting parameters for at least one of a) an ultrasonic device, b) delivery parameters for a medicine delivery device, or c) temperature control parameters for a temperature control device (157).

24. The method of claim 22, further comprising implementing the artificial intelligence in an analysis module which receives the data, the analysis module conducting an FEM analysis to simulate an influence of the shock waves at a cellular level, and outputting an effective treatment based for the setting parameters of the device (10; 20; 80; 90) for extracorporeal shock wave therapy.

25. A computer program fixed in a tangible medium comprising program code for performing the steps of the method according to claim 22 for execution by the processor unit.

26. The device for extracorporeal shock wave therapy according to claim 1, wherein the capacitor discharge resonant circuit has the discharge frequency distribution with the maximum being below 300 KHz.

27. The device for extracorporeal shock wave therapy according to claim 1, wherein the capacitor discharge resonant circuit has the discharge frequency distribution with the maximum being below 250 kHz.

28. The device for extracorporeal shock wave therapy according to claim 1, wherein the capacitor discharge resonant circuit has the discharge frequency distribution with the maximum being below 200 KHz.

29. The device for extracorporeal shock wave therapy according to claim 1, wherein the capacitor discharge resonant circuit has the discharge frequency distribution with the maximum being below 150 KHz.

30. The device for extracorporeal shock wave therapy according to claim 1, wherein the capacitor discharge resonant circuit has the discharge frequency distribution with the maximum being below 100 KHz.

31. The device for extracorporeal shock wave therapy according to claim 1, wherein the at least one shock wave generator (12) is an electrohydraulic shock wave generator.

32. A method for the treatment of Alzheimer's disease, comprising:
providing the device for extracorporeal shock wave therapy according to claim 1,
placing the at least one shock wave applicator (3) on a patient's head, and
operating the shock wave generator (12) to apply shock or pressure waves to the patient's head.

33. The device for extracorporeal shock wave therapy according to claim 4, wherein the coaxial high-voltage cable is longer than 2.0 m.

34. The device for extracorporeal shock wave therapy according to claim 4, wherein the coaxial high-voltage cable is longer than 3.0 m.

35. The device for extracorporeal shock wave therapy according to claim 4, wherein the coaxial high-voltage cable is longer than 5.0 m.

36. The device for extracorporeal shock wave therapy according to claim 4, wherein the coaxial high-voltage cable is longer than 10.0 m.

37. The device (10; 20; 80; 90) for extracorporeal shock wave therapy according to claim 2, wherein the frequency control unit (11) is configured to adjust the electrohydraulic shock wave generator (12) so that the capacitor discharge resonant circuit has the discharge frequency distribution that has the maximum below 300 KHz.

38. The device (10; 20; 80; 90) for extracorporeal shock wave therapy according to claim 2, wherein the frequency control unit (11) is configured to adjust the electrohydraulic shock wave generator (12) so that the capacitor discharge resonant circuit has the discharge frequency distribution that has the maximum below 250 KHz.

39. The device (10; 20; 80; 90) for extracorporeal shock wave therapy according to claim 2, wherein the frequency control unit (11) is configured to adjust the electrohydraulic shock wave generator (12) so that the capacitor discharge resonant circuit has the discharge frequency distribution that has the maximum below 200 kHz.

40. The device (10; 20; 80; 90) for extracorporeal shock wave therapy according to claim 2, wherein the frequency control unit (11) is configured to adjust the electrohydraulic shock wave generator (12) so that the capacitor discharge resonant circuit has the discharge frequency distribution that has the maximum below 150 KHz.

41. The device (10; 20; 80; 90) for extracorporeal shock wave therapy according to claim 2, wherein the frequency control unit (11) is configured to adjust the electrohydraulic shock wave generator (12) so that the capacitor discharge resonant circuit has the discharge frequency distribution that has the maximum below 100 KHz.

42. The device for extracorporeal shock wave therapy according to claim 6, wherein the shock wave that has left the shock wave applicator (3) has frequency bands whose dominant maxima are in the range between 80 KHz and 850 KHz.

43. The device for extracorporeal shock wave therapy according to claim 42, wherein the shock wave that has left the shock wave applicator (3) has frequency bands whose dominant maxima are in a range between 80 KHz and 450 KHz.

44. The device for extracorporeal shock wave therapy according to claim 42, wherein the shock wave that has left the shock wave applicator (3) has frequency bands whose dominant maxima are in a range between 80 kHz and 350 kHz.

45. The device for extracorporeal shock wave therapy according to claim 15, wherein the discharge resonant circuit has a capacitor with a charging voltage of between 1 kV and 7.5 kV.

46. The device for extracorporeal shock wave therapy according to claim 15, wherein the discharge resonant circuit has a capacitor and an energy of the shock wave device stored in the capacitor is 0.5 J to 5 J.
